(19) United States
(12) Patent Application Publication
(10) Pub. No.: US 2024/0165110 A1
(43) Pub. Date: May 23, 2024

(54) SELECTIVE AND NON-SELECTIVE OPIOID RECEPTOR FUNCTIONAL ANTAGONISTS AND METHODS RELATED THERETO FOR TREATMENT OF ADDICTION, OPIOD DEPENDENCE, AND NEUROPATHIC PAIN

(71) Applicant: Texas Tech University System, Lubbock, TX (US)

(72) Inventors: Nadezhda GERMAN, Amarillo, TX (US); Volker NEUGEBAUER, Lubbock, TX (US); Mohammad Anwar HOSSAIN, Amarillo, TX (US); Thomas ABBRUSCATTO, Amarillo, TX (US)

(73) Assignee: TEXAS TECH UNIVERSITY SYSTEM, Lubbock, TX (US)

(21) Appl. No.: 18/420,655

(22) Filed: Jan. 23, 2024

Related U.S. Application Data
(63) Continuation-in-part of application No. 17/290,987, filed on May 3, 2021.
(60) Provisional application No. 62/928,008, filed on Oct. 30, 2019.
(60) Provisional application No. 62/755,186, filed on Nov. 2, 2018.

(51) Int. Cl. A61K 31/4985; C07D 487/04

(52) U.S. Cl. CPC A61K 31/4985; C07D 487/04

(57) ABSTRACT

Disclosed is a composition and method for a therapeutic treatment that is able to combat certain conditions such as addiction, alcohol dependence, opioid abuse treatment, neurological disorders, neuropathic pain, fibromyalgia, and combinations thereof. The compounds act by acting as selective antagonist to the kappa (κ) opioid receptor. Further, disclosed compounds are an analog compound that can act as an antagonist to one or more opioid receptors. When present, these compounds lead to the inhibition of conditions, providing increased performance over known treatments. The disclosed compounds also shows the ability to cross the blood-brain-barrier in a highly efficient manner. The disclosed compounds are shown to be effective in the nanomolar range.

### CROSS-REFERENCE TO RELATED APPLICATIONS

This application is a continuation-in-part of U.S. patent application Ser. No. 17/290,987, filed May 3, 2021, entitled “NOVEL SELECTIVE KAPPA OPIOID RECEPTOR ANTAGONISTS AND METHODS RELATED THERETO FOR TREATMENT OF ADDICTION AND NEUROPATHIC PAIN”, which is the 35 U.S.C § 371 national application of PCT Application No. PCT/US2019/59524, filed on Nov. 1, 2019, entitled “NOVEL SELECTIVE KAPPA OPIOID RECEPTOR ANTAGONISTS AND METHODS RELATED THERETO FOR TREATMENT OF ADDICTION AND NEUROPATHIC PAIN”, which claims priority to U.S. Patent Application Ser. No. 62/928,008, filed on Oct. 30, 2019, entitled “NOVEL SELECTIVE KAPPA OPIOID RECEPTOR ANTAGONISTS AND METHODS RELATED THERETO FOR TREATMENT OF ADDICTION AND NEUROPATHIC PAIN”, and U.S. Patent Application Ser. No. 62/755,186, filed on Nov. 2, 2018, entitled “NOVEL SELECTIVE KAPPA OPIOID RECEPTOR ANTAGONISTS AND METHODS RELATED THERETO FOR TREATMENT OF ADDICTION AND NEUROPATHIC PAIN”, all of which are hereby incorporated herein by reference in its entirety for all purposes.

This application includes material that is subject to copyright protection. The copyright owner has no objection to the facsimile reproduction by anyone of the patent disclosure, as it appears in the Patent and Trademark Office files or records, but otherwise reserves all copyright rights whatsoever.

### TECHNICAL FIELD

The present invention relates in general to the field of therapeutic treatment. In particular, the present invention provides for a novel class of chemical compounds with selective and non-selective kappa opioid receptor antagonist properties. The disclosed compounds have potential to be used in patients with certain opioid and substance abuse as well as chronic pain.

### STATEMENT OF FEDERALLY FUNDED RESEARCH

None.

### BACKGROUND OF THE DISCLOSURE

Opioid receptors are inhibitory receptors with opioids as ligands. Opioid receptors are widely distributed in the brain and are also found throughout the spinal cord. There are 3 major subtypes of opioid receptors know as delta, kappa, and mu opioid receptors. These receptors are the targets of opium derived drugs known as opioids. Opioids are used for pain relief and anesthesia. Since opioids can produce strong feelings of euphoria, they are commonly used in a recreational setting, and are subject to abuse and addiction. In 2014 almost 2 million Americans abused and were dependent on prescription opioids, and in 2016 64,000 people died of an overdose of opioids. There is a major need for solutions to the increase in opioid usage.

Opioid receptor antagonists are a class of drugs that work by antagonizing or inhibiting the function of opioid receptors. These drugs work by competitively binding to the ligand receptor site of opioid receptors. This competitive inhibition does not allow opioid drugs to bind and thus, they have no effect. Certain opioid antagonists have such a high affinity for opioid receptors that they will displace the opioid drug from the receptor reversing its effect. There are several opioid antagonists currently used in the clinical setting. Naloxone and naltrexone are among the most common and are effective at treating opioid overdose and mitigating the effects of opioids and may be used to treat opioid dependency. However, due to limitation and drawbacks of current treatments, including side effects, there remains a need in the art to develop superior alternatives to current drugs on the market.

Moreover, opioids represent the most extensive category of abused substances in the United States, and the number of fatalities caused by these drugs exceeds those associated with all other drug overdoses combined. The administration of naltrexone, a potent pan-opioid receptor antagonist, to an individual dependent on opioids can trigger opioid withdrawal and induce severe side effects. There is a pressing demand for opioid antagonists that do not produce opioid withdrawal effects.

Drug addiction is a chronic and relapsing disease of the central nervous system (CNS), resulting in compulsive seeking and use despite physical, social, and psychological harm to the user. It directly contributes to an estimated annual cost of over $190 billion from lost work productivity, health care costs, and crime in the United States. Prescription opioid painkillers and heroin represent the largest class of abused agents in the U.S., and deaths from these drugs now exceed all other drug overdose deaths. The three approved medications for opioid addiction, methadone, buprenorphine, and naltrexone, are opioid receptor ligands. Methadone and buprenorphrine, full and partial Mu Opioid Receptor (MOR) agonists respectively. Primarily due to their relatively extended half-life, these compounds can induce dependence on their own, and discontinuing their use can trigger withdrawal symptoms, potentially leading to opioid relapse. Naltrexone, a potent non-selective opioid receptor antagonist, can precipitate opioid withdrawal symptoms when administered to an opioid-dependent individual, resulting in severe side effects such as agitation, nausea, vomiting, pain, and diarrhea. Recent studies have highlighted the potential of selective Kappa Opioid Receptor (KOR) antagonists in reversing or preventing the formation of addiction linked to the use of traditional opioid analgesics, which are primarily MOR agonists. These findings suggest a promising avenue for treating opioid addiction with a lower risk of dependence and relapse.

Thus, a non-selective opioid or selective kappa-opioid functional antagonist may be useful therapeutically as an opioid withdrawal medication.

### SUMMARY OF THE DISCLOSURE

The present invention addresses failings in the art by providing class of selective kappa opioid receptor antagonists, particularly, two structurally dissimilar opioid ligands that selectively inhibit the kappa opioid receptor (KOR). Literature has suggested that KOR antagonists may be helpful in the treatment of addiction, withdrawal symptoms and pain, and many psychological disorders. There are currently several different KOR antagonists that are being studied and participating in clinical trials. In addition, the present invention provides for methods for a therapeutic treatment that utilizes such compounds as opioid receptor antagonists.

The novel compounds of the present invention were unexpectedly found to be selective antagonists of kappa opioid receptors, while exhibiting little or no binding at delta or mu receptors. Thus, in one aspect, the present invention provides a pharmaceutical formulation comprising an effective amount of a compound (see FIG. 1A) sufficient as a selective antagonist of the kappa-opioid receptor.

In another aspect, the present invention provides a pharmaceutical formulation comprising an effective amount of a compound Formula II, (see FIG. 1B) sufficient as a selective antagonist of the kappa-opioid receptor.

In another aspect, the present invention provides a pharmaceutical formulation comprising an effective amount of a compound of the novel class of Formula III (see FIG. 2A) sufficient as a selective antagonist of the kappa-opioid receptor.

In another aspect, the present invention provides a pharmaceutical formulation comprising an effective amount of a compound of the novel class of Formula IV (see FIG. 2B) sufficient as a selective antagonist of the kappa-opioid receptor. R2 is a chemical group selected from a group consisting of: hydrogen, hydroxy, alkyloxy, alkyl ester, amine, alkylamine, dialkylamine, thio, thioalkyl, and alkyl ethers. R3 is a chemical group selected from a group consisting of: hydrogen, benzyl, substituted benzyl, methyl, alkyl carbamate, alkyl, prenyl, and cycloalkyl. R4 is a chemical group selected from a group consisting of: hydrogen, alkyl, acetyl, cycloalkyl, benzyl, and substituted benzyl. R5 is a chemical group selected from a group consisting of: alkyl, alkyl ester of carboxylic acid, alkyl ether, alkylamide, amine, monoalkylamine, dialkyl amine, cycloalkyl, and hydrogen. R6 is a chemical group selected from a group consisting of: hydrogen, alkyl ether, halogen, alkyl, amine, monoalkylamine, and dialkyl amine.

The present invention also provides a method for blocking kappa opioid receptors in mammalian tissue comprising contacting said receptors in vivo or in vitro with an effective amount of the compound of any of Formulas I-IV, preferably in combination with a pharmaceutically acceptable vehicle. Therefore, the compound of any of Formulas I-IV which exhibit kappa receptor antagonist activity may also be therapeutically useful in conditions where selective blockage of kappa receptors is desired. This includes blockage of the appetite response, blockage of paralysis due to spinal trauma and a variety of other physiological activities that may be mediated through kappa receptors. The novel compounds of the present invention also show the ability to cross the blood-brain-barrier (BBB) where many opioid receptors are present. This novel class of compounds has the potential to be a powerful new treatment to combat conditions such as general opioid abuse, addiction, alcohol dependence, neuropathic pain, and other chronic pain conditions. The novel class of compounds are further able to serve as a pain reducer and do not induce addiction. Moreover, when giving in combination with current opioid analgesics, they significantly decrease opioid-seeking behavior.

It is therefore and object of the present invention to provide the compound of any of Formulas I-IV, VI-XIII, XV-XXXVII, XXXVIII-LIII, and LIV-LXVI. In one aspect of the present invention, said compounds have immunosuppressive capabilities. The KOR antagonist compounds of the present invention offer a new class of selective antagonists that are structurally different from known compounds. In addition, it may offer an increase in the duration of action of the drug since it exhibits a longer half-life.

In another aspect, said compound is capable of having at least 50% of the administered amount cross the blood-brain barrier (BBB) of a patient. In another aspect, said compound is capable of having at least 80% of the administered amount cross the BBB of a patient. In yet another aspect, said compound does not cross the BBB of a patient, instead acting peripherally to treat cancer chemotherapy-induced pain and allodynia. The compound of the present invention is effective to treat addiction, alcohol dependence, opioid abuse treatment, neurological disorders, neuropathic pain, and combinations thereof, and is capable of inhibition of the CNS receptor known as the kappa (κ) opioid receptor. The compound of the present invention is further effective to block or reduce the tolerance of said mammal to an opioid receptor agonist, such as morphine, methadone, codeine, diacetyl morphine, morphine-N-oxide, oxymorphone, oxycodone, hydromorphone, hydrocodone, meperidine, heterocodeine, fentanyl, sufentanil, levo-acetylmethadol, alfentanil, levorphanol, tilidine, diphenoxylate, hydroxymorphone, noroxymorphone, metopon, propoxyphene, and the pharmaceutically acceptable salts thereof.

In another aspect of the present invention, a method is provided for treating a disorder selected from the group consisting of addiction, alcohol dependence, opioid abuse treatment, neurological disorders, neuropathic pain, and combinations thereof, comprising administering to a patient a therapeutically effective amount of a compound of any of Formulas I-IV, Formulas VI-XIII, Formulas XV-XXXVII, Formulas XXXVIII-LIII, Formulas LIV-LXVI, or a pharmaceutically acceptable salt thereof or isotopic variants thereof, stereoisomers or tautomers thereof. In another aspect a method for of treating a mammal is provided comprising the step of: administering to a patient a therapeutically effective amount of a compound of any of Formulas I-IV, Formulas VI-XIII, Formulas XV-XXXVII, Formulas XXXVIII-LIII, Formulas LIV-LXVI, or a pharmaceutically acceptable salt thereof or isotopic variants thereof, stereoisomers or tautomers thereof, wherein said therapeutically effective amount is effective as an antagonist to one or more opioid receptors.

In another aspect the compounds of the present invention comprise an aqueous solution and one or more pharmaceutically acceptable excipients, additives, carriers or adjuvants. In another aspect the compound further comprises one or more excipients, carriers, additives, adjuvants, or binders in a tablet or capsule.

In another aspect a compound of the present invention is administered via an oral, intraperitoneal, intravascular, peripheral circulation, subcutaneous, intraorbital, ophthalmic, intraspinal, intracisternal, topical, infusion, implant, aerosol, inhalation, scarification, intracapsular, intramuscular, intranasal, buccal, transdermal, pulmonary, rectal, or vaginal route.

The present invention further addresses failings in the art by providing a non-selective opioid receptor ligand with similar affinity profiles to MOR, Delta Opioid Receptor (DOR), and KOR. In some embodiments, the present invention includes a compound is based on a tricyclic system containing a diketopiperazine (DKP) moiety. There present invention further discloses a series of analogs with modifications around the DKP scaffold were designed to optimize the observed affinity of the non-selective opioid receptor ligand.

Specifically, the present invention addresses failings in the art by providing compositions of derivatives capable of serving as a non-selective opioid receptor antagonist, as well as methods for a therapeutic treatment that utilizes such compounds as opioid receptor antagonists.

Thus, in one aspect, the present invention provides a pharmaceutical formulation comprising an effective amount of a compound (see FIG. 14), a tricyclic DKP-based analog, sufficient as a non-selective opioid receptor ligand.

In some aspects, the novel compounds of the present invention are derivatives with the presence of a disulfide bridge and function via generation of reactive oxygen species (ROS) and mixed disulfide formation. The novel compounds of the present invention also show the ability to cross the blood-brain-barrier (BBB) where many opioid receptors are present. This novel class of compounds has the potential to be a powerful new treatment to combat conditions such as general opioid abuse, alcohol dependence, neuropathic pain, fibromyalgia, and other chronic pain conditions, including but not limited to pain associated with various cancers.

It is therefore and object of the present invention to provide derivative compounds of Formula V. In one aspect of the present invention, the opioid antagonist compounds of the present invention offer a new class of antagonists that are structurally different from known compounds. In addition, it may offer an increase in the duration of action of the drug since such derivative compounds exhibit a longer half-life.

In one aspect, the present invention provides a compound of substituted derivatives, or a pharmaceutically acceptable salt thereof.

In one aspect, the present invention provides a pharmaceutical composition including a therapeutically effective amount of the substituted derivatives, or a pharmaceutically acceptable salt thereof, isotopic variants, stereoisomers or tautomers thereof.

In one aspect, the present invention provides a pharmaceutical composition including an effective amount of the substituted derivatives sufficient as a non-selective antagonist of opioid receptors.

In one aspect, the present invention is effective to treat alcohol dependence, opioid abuse treatment, neurological disorders, neuropathic pain, and fibromyalgia.

In one aspect, the present invention is effective to block or reduce the tolerance of said human to an opioid receptor agonist.

### BRIEF DESCRIPTION OF THE DRAWINGS

The foregoing and other objects, features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1A depicts the chemical structure for Formula I.

FIG. 1B depicts the chemical structure for Formula II.

FIG. 2A depicts the chemical structure for Formula III.

FIG. 2B depicts the chemical structure for Formula IV.

FIG. 3 depicts data relating to rodent-based audible vocalizations for Formula II.

FIG. 4 depicts data relating to rodent-based ultrasonic vocalizations for Formula II.

FIG. 5 depicts data relating to rodent-based Electronic von Frey measurements for Formula II.

FIG. 6 depicts data relating to rodent-based withdraw threshold measurements for Formula II.

FIG. 7 depicts a synthesis schematic of Formula I and Formula II of the present invention.

FIG. 8 depicts data relating to rodent-based audible vocalizations for Formula IV.

FIG. 9 depicts data relating to rodent-based ultrasonic vocalizations for Formula IV.

FIG. 10 depicts data relating to rodent-based Electronic von Frey measurements for Formula IV.

FIG. 11 depicts data relating to rodent-based audible vocalizations for Formula IV.

FIG. 12 depicts data relating to rodent-based ultrasonic vocalizations for Formula IV.

FIG. 13 depicts a synthesis schematic of Formula V of the present invention.

FIG. 14 depicts the chemical structure for Formula V.

FIG. 15A depicts data relating to basolateral transport in parallel artificial permeability assays for Formula V.

FIG. 15B depicts data relating to basolateral transport across an in-vitro bEnd-3 monolayer model for Formula V.

FIG. 16 depicts data relating to bidirectional transport in Caco-2 cell monolayers for Formula V.

FIG. 17 depicts data relating to the rodent-based in-vivo pharmacokinetic profile of Formula V.

FIG. 18 depicts data relating to the maximum possible antinociceptive effect of compounds, including Formula V, on rodents.

FIG. 19A depicts data relating to the stimulus intensity of compounds, including Formula V, on paclitaxel-induced mechanical allodynia.

FIG. 19B depicts data relating to the stimulus intensity of compounds, including Formula V, on paclitaxel-induced mechanical allodynia.

FIG. 20 depicts the chemical structure for Formula VI.

FIG. 21 depicts the chemical structure for Formula VII.

FIG. 22 depicts the chemical structure for Formula VIII.

FIG. 23 depicts the chemical structure for Formula IX.

FIG. 24 depicts the chemical structure for Formula X.

FIG. 25 depicts the chemical structure for Formula XI.

FIG. 26 depicts the chemical structure for Formula XII.

FIG. 27 depicts the chemical structure for Formula XIII.

FIG. 28 depicts the chemical structure for Formula XIV.

FIGS. 29A-29W depict a series of “open chain” analogs. FIG. 29A depicts the chemical structure for Formula XV. FIG. 29B depicts the chemical structure for Formula XVI. FIG. 29C depicts the chemical structure for Formula XVII. FIG. 29D depicts the chemical structure for Formula XVIII. FIG. 29E depicts the chemical structure for Formula XIX. FIG. 29F depicts the chemical structure for Formula XX. FIG. 29G depicts the chemical structure for Formula XXI. FIG. 29H depicts the chemical structure for Formula XXII. FIG. 29I depicts the chemical structure for Formula XXIII. FIG. 29J depicts the chemical structure for Formula XXIV. FIG. 29K depicts the chemical structure for Formula XXV. FIG. 29L depicts the chemical structure for Formula XXVI. FIG. 29M depicts the chemical structure for Formula XXVII. FIG. 29N depicts the chemical structure for Formula XXVIII. FIG. 29O depicts the chemical structure for Formula XXIX. FIG. 29P depicts the chemical structure for Formula XXX. FIG. 29Q depicts the chemical structure for Formula XXXI. FIG. 29R depicts the chemical structure for Formula XXXII. FIG. 29S depicts the chemical structure for Formula XXXIII. FIG. 29T depicts the chemical structure for Formula XXXIV. FIG. 29U depicts the chemical structure for Formula XXXV. FIG. 29V depicts the chemical structure for Formula XXXVI. FIG. 29W depicts the chemical structure for Formula XXXVII.

FIGS. 30A-30P depict a series of “closed chain” analogs. FIG. 30A depicts the chemical structure for Formula XXXVIII. FIG. 30B depicts the chemical structure for Formula XXXIX. FIG. 30C depicts the chemical structure for Formula XL. FIG. 30D depicts the chemical structure for Formula XLI. FIG. 30E depicts the chemical structure for Formula XLII. FIG. 30F depicts the chemical structure for Formula XLIII. FIG. 30G depicts the chemical structure for Formula XLIV. FIG. 30H depicts the chemical structure for Formula XLV. FIG. 30I depicts the chemical structure for Formula XLVI. FIG. 30J depicts the chemical structure for Formula XLVII. FIG. 30K depicts the chemical structure for Formula XLVIII. FIG. 30L depicts the chemical structure for Formula XLIX. FIG. 30M depicts the chemical structure for Formula L. FIG. 30N depicts the chemical structure for Formula LI. FIG. 30O depicts the chemical structure for Formula LII. FIG. 30P depicts the chemical structure for Formula LIII.

FIGS. 31A-31M depict a series of exemplary embodiments of analogs that are structured based on the chemical structure of Formula VI. FIG. 31A depicts the chemical structure for Formula LIV. FIG. 31B depicts the chemical structure for Formula LV. FIG. 31C depicts the chemical structure for Formula LVI. FIG. 31D depicts the chemical structure for Formula LVII. FIG. 31E depicts the chemical structure for Formula LVIII. FIG. 31F depicts the chemical structure for Formula LIX. FIG. 31G depicts the chemical structure for Formula LX. FIG. 31H depicts the chemical structure for Formula LXI. FIG. 31I depicts the chemical structure for Formula LXII. FIG. 31J depicts the chemical structure for Formula LXIII. FIG. 31K depicts the chemical structure for Formula LXIV. FIG. 31L depicts the chemical structure for Formula LXV. FIG. 31M depicts the chemical structure for Formula LXVI.

### DETAILED DESCRIPTION OF THE DISCLOSURE

While the making and using of various embodiments of the present invention are discussed in detail below, it should be appreciated that the present invention provides many applicable inventive concepts that can be embodied in a wide variety of specific contexts, goods, or services. The specific embodiments discussed herein are merely illustrative of specific ways to make and use the disclosure and do not delimit the scope of the disclosure.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The present invention will now be described more fully hereinafter with reference to the accompanying drawings, which form a part hereof, and which show, by way of illustration, specific example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, compositions, or systems. Accordingly, embodiments may, for example, take the form of methods, compositions, compounds, materials, or any combination thereof. The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or a direct connection. Thus, if a first device couples to a second device, that connection may be through a direct connection or through an indirect connection via other devices and connections.

The term “treating” refers to reversing, alleviating, or inhibiting the progress of a disease, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a compound or composition of the present invention to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

The terms “subject”, “individual”, or “patient” are used interchangeably herein and refer to an animal preferably a warm-blooded animal such as a mammal. Mammal includes without limitation any members of the Mammalia. In general, the terms refer to a human. The terms also include domestic animals bred for food or as pets, including equines, bovines, sheep, poultry, fish, porcines, canines, felines, and zoo animals, goats, apes (e.g. gorilla or chimpanzee), and rodents such as rats and mice.

Unless otherwise indicated, all numbers expressing quantities of ingredients, reaction conditions, and so forth used in the specification are to be understood as being modified in all instances by the term “about.” Accordingly, unless indicated to the contrary, the numerical parameters set forth in this specification are approximations that can vary depending upon the desired properties sought to be obtained by the presently disclosed subject matter.

The compounds Formula I and Formula II can exist in unsolvated as well as solvated forms with pharmaceutically acceptable solvents such as water, ethanol, and the like. The solvated forms may be considered equivalent to the unsolvated forms for the purposes of the present invention.

“Therapeutically effective amount” relates to the amount or dose of an active compound of either Formula I, Formula II, Formula VII, Formula VIII, Formula IX, Formula X, Formula XIII, or a composition comprising the same, that will lead to one or more desired effects, in particular, one or more therapeutic effects, more particularly beneficial effects. A therapeutically effective amount of a substance can vary according to factors such as the disease state, age, sex, and weight of the subject, and the ability of the substance to elicit a desired response in the subject. A dosage regimen may be adjusted to provide the optimum therapeutic response (e.g. sustained beneficial effects). For example, several divided doses may be administered daily or the dose may be proportionally reduced as indicated by the exigencies of the therapeutic situation.

The term “pharmaceutically acceptable” salt, carrier, excipient, or vehicle refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbents that may be needed in order to prepare a particular composition. Examples of carriers etc. include but are not limited to a salt formulation, saline, buffered saline, dextrose, water, glycerol, ethanol, and combinations thereof. The use of such media and agents for an active substance is well known in the art.

In one embodiment of the present invention, each of Formulas I-IV are compounds with the ability to penetrate the blood brain barrier and inhibit kappa opioid receptors selectively at the level of up to 90% at 10 uM concentration. In some embodiments, these compounds are being effluxed from the brain and exert their pharmacological effects peripherally, rather than centrally. In such an embodiment, this peripheral action can be particularly useful in treating cancer chemotherapy-induced pain. Active analogs also have no signs of toxicity when administered to animals (rats) and have moderate to excellent enzymatic stability, suggesting its use in oral form, when applicable. In vivo studies have shown that the compounds of the present invention have reduced pain markers by 40-50% and this result is observed starting 30 minutes after administration of compounds.

Converging evidence from recent research strongly suggests that enhanced kappa opioid receptor (KOR) signaling in brain circuits promotes aversiveness and hypersensitivity in chronic pain, whereas systemically administered kappa-antagonists have analgesic effects. Moreover, kappa antagonists were shown to inhibit the addictive effect of mu opioid receptor (MOR) agonists and alleviate symptoms of opioid-induced addiction; attenuate alcohol seeking and withdrawal anxiety. Current literature suggests that includes clinical and basic research reports a strong connection between post-traumatic stress disorder and opioid/alcohol abuse. Moreover, evidence supports that similar association exists between brain injury and substance abuse. In this case, an overdose often leads to a higher incidence of brain injury, and patients with traumatic brain disorders are more likely to develop a substance abuse disorder. Therefore, KOR antagonists are envisioned to have significant clinical potential as a non-addictive pain management tool and as modulators of drug-seeking behavior.

Opioid receptors belong to the large superfamily of seven transmembrane-spanning G protein-coupled receptors (GPCRs). As a class, GPCRs are of fundamental physiological importance in mediating the actions of the majority of known neurotransmitters and hormones. They are activated endogenously produced opioid peptides and by exogenously administered opiate compounds. Many of these exogenous compounds are used for pain relief and anesthesia, and these, often, give off strong feelings of euphoria. This strong euphoria makes them a common drug that can be abused in a recreational setting. The effects of this opioid crisis led to more than 115 Americans death per day due to opioid related overdoses, devastating families and communities across the country.

The present invention presents three analog classes of compounds with the ability to penetrate the blood brain barrier (BBB) and inhibit kappa opioid receptors (KOR) selectively or have peripheral distribution and the ability to selectively inhibit KOR. The active analogs have shown little to no signs of toxicity in laboratory tests that have been performed in rats, and these analogs have shown strong enzymatic stability that suggests its possible use in oral form. These compounds are shown to work as pain reducers that do not induce addiction. They could replace existing opioid analgesics or could be given in combination with current opioid analgesics to significantly decrease opioid-seeking behavior. These active compounds could also work to solve depression, alcohol seeking behavior and opioid withdrawal symptoms.

The present invention thus presents two structurally novel KOR antagonists with non-opioid pharmacophores. These compounds were confirmed to inhibit kappa opioid receptors selectively, with Ki values of 274 nM and 924 nM, with up to 90% of KOR being inhibited. In addition, BBB permeability of these chemical ligands is confirmed in vitro, and lack of toxicity in rat neurons and bEnd3 cells. Additional experimentation presents pharmacological activity using the well-established spinal nerve ligation (SNL) model of neuropathic pain in rats, where inhibitory effects of KOR antagonism were found on emotional-affective pain responses (audible and ultrasonic vocalization) and hypersensitivity (electronic von Frey test).

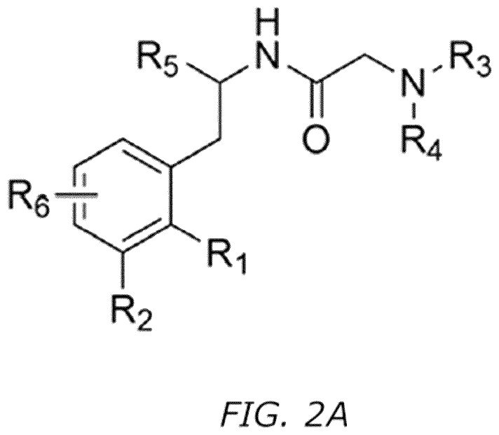

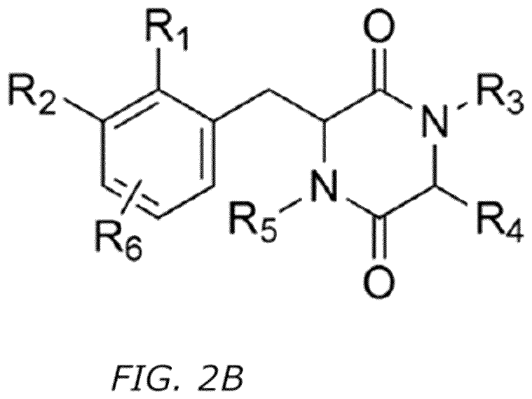

Turning to FIG. 2A, Formula III of the present invention is provided with the various R-groups. In one embodiment, R1 is a chemical group selected from a group consisting of: hydrogen, halogen, nitro, alkyl, aliphatic, cycloalkyl, trifluoroalkyl, substituted phenyl, carboxylic acid, alkyl ester of carboxylic acid, and acetyl.

In one embodiment, R2 is a chemical group selected from a group consisting of: hydrogen, halogen, hydroxy, alkyloxy, alkyl ester, amine, alkylamine, dialkylamine, thio, thioalkyl, and alkyl.

In one embodiment, R3 is a chemical group selected from a group consisting of: hydrogen, benzyl, substituted benzyl, methyl, alkyl carbamate, alkyl, prenyl, and cycloalkyl.

In one embodiment, R4 is a chemical group selected from a group consisting of: hydrogen, alkyl, acetyl, cycloalkyl, benzyl, and substituted benzyl.

In one embodiment, R5 is a chemical group selected from a group consisting of: alkyl, alkyl ester of carboxylic acid, alkyl ether, alkylamide, amine, monoalkylamine, dialkyl amine, cycloalkyl, and hydrogen.

In one embodiment, R6 is a chemical group selected from a group consisting of: hydrogen, alkyl ether, halogen, alkyl, amine, monoalkylamine, and dialkyl amine.

FIG. 2B presents Formula IV of the present invention, together with the representative R-groups.

In one embodiment, R1 is a chemical group selected from a group consisting of: hydrogen, halogen, nitro, alkyl, aliphatic, cycloalkyl, trifluoroalkyl, substituted phenyl, carboxylic acid, alkyl ester of carboxylic acid, and acetyl.

In one embodiment, R2 is a chemical group selected from a group consisting of: hydrogen, halogen, hydroxy, alkyloxy, alkyl ester, amine, alkylamine, dialkylamine, thio, thioalkyl, and alkyl.

In one embodiment, R3 is a chemical group selected from a group consisting of: hydrogen, benzyl, substituted benzyl, methyl, alkyl carbamate, alkyl, prenyl, and cycloalkyl.

In one embodiment, R4 is a chemical group selected from a group consisting of: hydrogen, alkyl, acetyl, cycloalkyl, benzyl, and substituted benzyl.

In one embodiment, R5 is a chemical group selected from a group consisting of: alkyl, alkyl ester of carboxylic acid, alkyl ether, alkylamide, amine, monoalkylamine, dialkyl amine, cycloalkyl, and hydrogen.

In one embodiment, R6 is a chemical group selected from a group consisting of: hydrogen, alkyl ether, halogen, alkyl, amine, monoalkylamine, and dialkyl amine.

In another embodiment, both Formula III and Formula IV are capable of maintaining KOR antagonist properties in the nanomolar range.

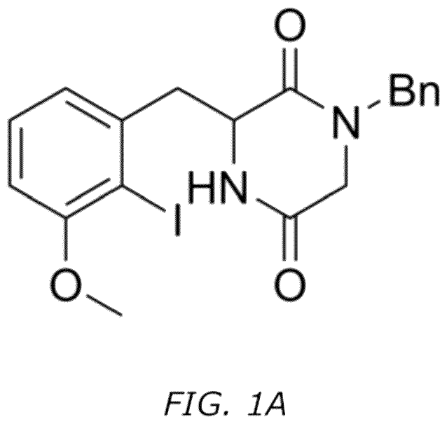

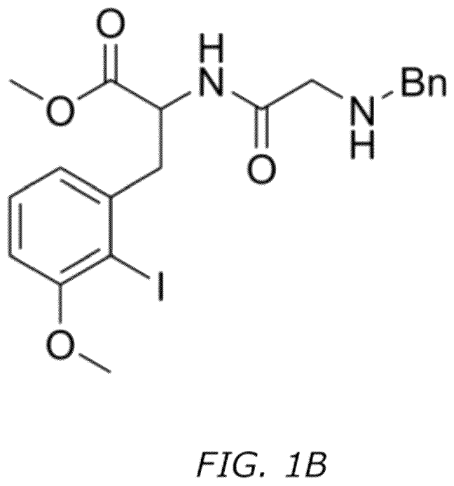

Turning to FIG. 1A, Formula I (AH-3-99) of the present invention is provided. The inhibition of the KOR is quantified in Table 1, as is the inhibition constant (Ki) value. FIG. 1B presents Formula II (AH-3-193) of the present invention. The inhibition of the KOR is quantified in Table 1, as is the inhibition constant (Ki) value. Both Formula I and Formula II are capable of maintaining KOR antagonist properties in the nanomolar range.

These compounds of the present invention are shown to inhibit pain response 20-40% (when different pain markers are assessed). Additionally, PK analysis was performed of the class II parent compound (Formula IV) in rats. The observed data are set forth in Table 1, below.

TABLE 1
Formula IV pK data from in vivo study.

| | |
|---|---|
| Injection: | i.p. 3 mg/kg |
| Plasma: | Cₘₐₓ = 263 ng/mL |
| | Tₘₐₓ = 15 mins |
| | AUClast - 12083 min ng/m |
| Brain: | Cmax = 172 ng/mL |
| | Tmax = 30 mins |
| | AUClast = 6562 min ng/m |
| BBB Permeability: | Brain to plasma ratio is 0.5:1 |

It is therefore an embodiment of the present invention to provide novel compounds of Formula I and Formula II, and derivatives thereof, or pharmaceutically acceptable salts thereof, which are presented for treating certain disorders in a patient, such as addiction, alcohol dependence, opioid abuse treatment, neurological disorders, and neuropathic pain. It is another embodiment of the present invention to provide pharmaceutically acceptable compositions comprising compounds of Formula I and Formula II, and a pharmaceutically acceptable carrier.

Another embodiment of the present invention relates to a method for providing neuroprotection in a mammal in need of such treatment. The method comprises administering to the mammal a therapeutically effective amount of a compound of any of Formulas I-IV or a pharmaceutically acceptable salt thereof. In another embodiment, the present invention provides a pharmaceutical composition comprising a therapeutically effective amount of a compound of the invention or a pharmaceutically acceptable salt thereof in combination with one or more pharmaceutically acceptable carriers. The composition is preferably useful for the treatment of the disease conditions described above.

Further, the present invention provides the use of a compound of Formulas I-IV or a pharmaceutically acceptable salt thereof, in the manufacture of a medicament for the treatment of the disease conditions described above.

In another embodiment, the compounds, compositions, and methods disclosed herein therefore may be utilized to prevent and/or treat a disease such as alcohol dependence, opioid abuse treatment, neurological disorders, neuropathic pain, and fibromyalgia, among other conditions mediated by one or more opioid receptors.

In an illustrative embodiment of the present invention, Formula I and Formula II, were evaluated for selective activity of the KOR. Turning to Table 2, inhibitory data is provided for the KOR. Inhibition data is provided as inhibitor constant, Ki, is the concentration required to produce half-maximum inhibition. This quantitative measure indicates how much of a particular drug or other substance (inhibitor) is needed to inhibit a given biological process (or component of a process, i.e. an enzyme, cell, cell receptor or microorganism) by half. The values are typically expressed as molar (micro- and nano-) concentrations.

TABLE 2
Inhibitory data relating to KOR inhibition of Formula
I and Formula II of the present invention.

| | Formula I | | Formula II | |
|---|---|---|---|---|
| | (AH-3-199) | | (AH-3-193) | |
| Ki | 924 | nM | 277 | nM |

| | | |
|---|---|---|
| Inhibition (%) | 74.9% | 86.5% |

In one embodiment, compound of the present invention Either Formula I or Formula II, are shown Table 1 to show inhibition with regard to various receptors, including opioid receptors μ (MOR), σ (DOR), and κ (KOR).

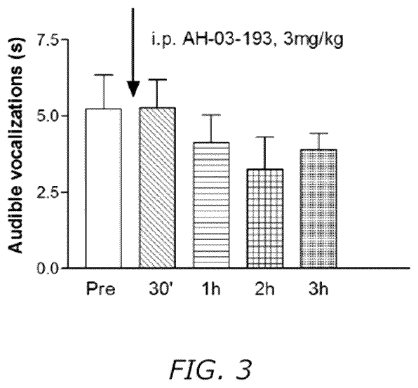

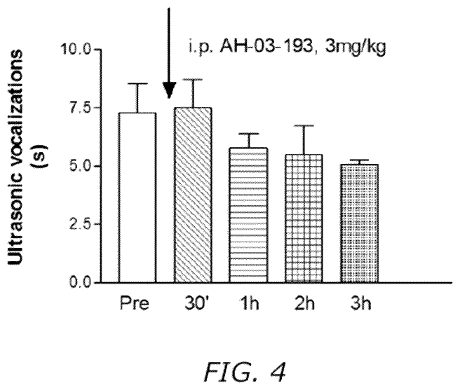

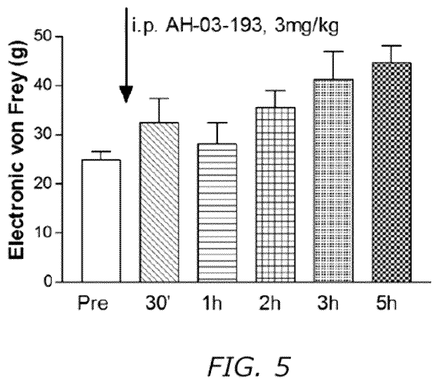

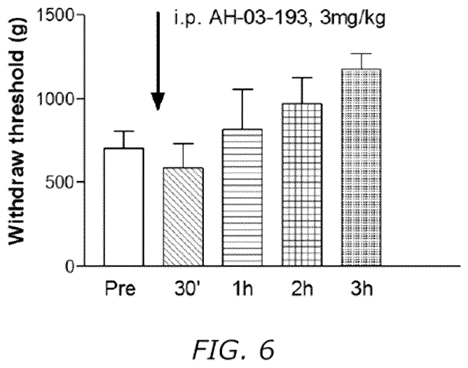

FIGS. 3-6 provides data related to certain pain indices in rodent studies, which include, audible vocalization(s) (FIG. 3), ultrasonic vocalizations (FIG. 4), electronic von Frey (FIG. 5), and withdraw threshold (FIG. 6). These studies have been considered to be valid, quantitative, reliable and convenient methods to measure pain-related behavior.

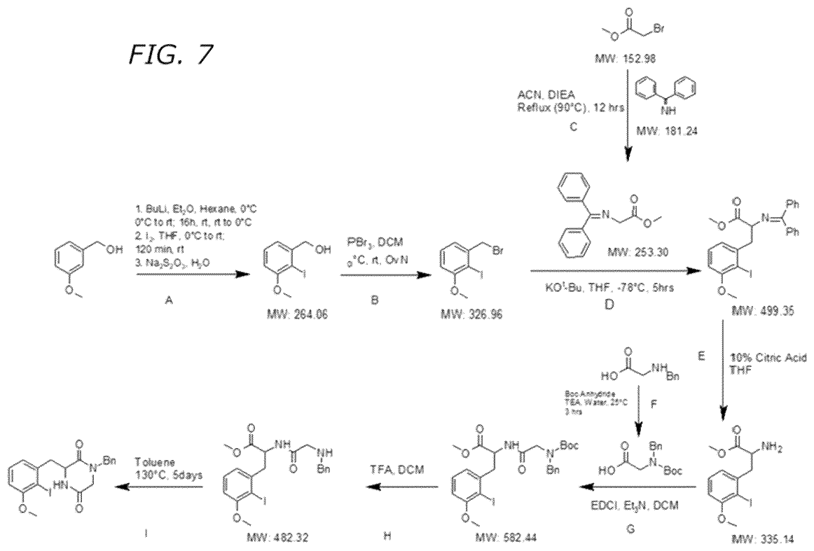

In another embodiment, the synthesis of Formula I and Formula II is as follows, in accordance with FIG. 7. FIG. 7—Step A. A stirred solution of alcohol (5.06 g, 36.6 mM) in a mixture of diethyl ether (61 mL) and hexane (183 mL) was cooled to 0° C. n-Butyllithium (2.5 M in hexanes, 32.3 mL, 80.7 mmol) was added dropwise and the resulting solution was warmed to room temperature, stirred overnight and then cooled to 0° C. A solution of iodine (14.0 g, 55.1 mmol) in THF (61 mL) was added. The resulting mixture was warmed to room temperature and stirred for 4 hours, followed by addition of 10% aqueous sodium thiosulfate. The organic portion was separated and the aqueous layer was extracted with the ethyl acetate. The combined organic extracts were washed with brine, dried over anhydrous magnesium sulfate, filtered and concentrated under reduced pressure. Product was purified by column chromatography using 3 to 17 ethyl acetate/petrol 40-60 eluent and further recrystallized using hexane/ethyl acetate mixture to afford the title compound as a white solid (37% for 2 steps).

FIG. 7—Step B. In an oven-dried round-bottomed flask, alcohol (6.4 g, 24.2 mmol) was dissolved in anhydrous dichloromethane (50 mL). At 0° C., PBr₃ (0.91 mL, 9.7 mmol, 0.4 equiv) was added slowly into the reaction flask, and the reaction mixture was stirred at 0° C. for 30 min, then at RT overnight. The reaction mixture was quenched with water (20 mL) and extracted with dichloromethane (3×20 mL). The combined organic phases were washed with water (2×50 mL), brine (50 mL) and dried over anhydrous sodium sulfate. The filtered solution was concentrated in vacuo to yield the crude product 1-(bromomethyl)-2-iodobenzene (91%) as white crystals, which was used without further purification.

FIG. 7—Step C. A solution of methyl bromoacetate (5.5 g, 35.9 mmol) in acetonitrile (40 mL) was treated with benzophenonimine (6.5 g, 35.8 mmol) and diisopropylethylamine (6.2 mL, 4.6 g, 35.6 mmol), and the mixture was then heated at reflux (90° C.) for 12 hours. After reaction completion, the mixture was cooled to room temperature, and most of the acetonitrile was removed in vacuo. The residue was partitioned between water (40 mL) and diethyl ether (60 mL) and the phases were separated. The organic portion was dried with MgSO₄, filtered, and concentrated in vacuo. Purification by flash column using ethyl acetate/hexane (10 to 15%) yielded product as white crystals (70%).

FIG. 7—Step D. KOtBu (0.484 g, 4.3 mmol) was suspended in THF (18 mL) and cooled to −78° C. The corresponding imine (0.916 g, 3.6 mmol) in THF (18 mL) was added to the suspension via cannula. After stirring for 30 min, a substituted benzyl bromide (1.3 g, 3.97 mmol) in THF (18 mL) was added to the reaction mixture via cannula. After 5 h the reaction solution was warmed to room temperature and water (40 mL) was added, followed by the removal of THF in vacuo. The aqueous solution was extracted with CH₂Cl₂ (3×20 mL) and the combined organics were washed with brine (40 mL), dried with Na₂SO₄, filtered and concentrated in vacuo. The crude material was purified by flash chromatography (5% Et₂O/benzene) to provide title compound as a light-yellow solid (47%).

FIG. 7—Step E. A 10% aq. solution of citric acid (32 mL) was added to a solution of imine (step D, 4.82 g, 9.65 mmol) in THF (97 mL) and allowed to stir for 16 h. Upon reaction completion, it was diluted with Et₂O (40 mL) and extracted with 1N aq. HCl (2×30 mL). The acidic solution was washed with Et₂O (2×30 mL) and basified with solid K₂CO₃. The basic aqueous solution was extracted with EtOAc (3×30 mL), washed with brine (50 mL), dried with MgSO₄, filtered and concentrated in vacuo to yield crude residue (90% yield), which was used in the next step without prior purification.

FIG. 7—Step F. A 500-mL round-bottom flask was charged with N-benzylglycine (10.0 mmol), a water solution (100 mL) containing triethylamine (4.18 mL, 30.0 mmol, 3 equiv.; FW: 101.19 g/mol), and a teflon-coated stir bar. Di-tert-butyl dicarbonate (2.18 g, 10.0 mmol, 1 equiv.; FW: 218.25 g/mol) was added and the solution was stirred for 3 h. The reaction mixture was poured into aqueous HCl (1N, 30 mL) and extracted with ethyl acetate (3×100 mL). The organic layer was washed with water (1×100 mL) and brine (2×50 mL), dried over MgSO₄, filtered, and concentrated under reduced pressure to obtain product as white solid (93%).

FIG. 7—Step G. To a solution of amine from step E (900 mg, 2.7 mmol), respective N-substituted amino acid (step F, 860 mg, 3.24 mmol), and EDCI (518 mg, 2.7 mmol) in CH₂Cl₂ (6 mL) was added Et₃N (0.5 mL) and the reaction was allowed to stir for 20 h, followed by dilution with CH₂Cl₂ (20 mL) and washed with 10% HCl in water (20 mL) then sat. aq. NaHCO₃ (2×20 mL). The combined aqueous washes were extracted with CH₂Cl₂ (2×20 mL) and the organics were combined and washed with brine (50 mL), dried over Na₂SO₄, filtered and concentrated in vacuo. The crude material was purified by flash chromatography (15:1 CH₂Cl₂:MeOH) to give dipeptide (83%).

FIG. 7—Step H. Dipeptide (from step G) was dissolved in CH₂Cl₂ (10 mL) and TFA (1.5 mL) was added. The reaction stirred overnight and the solvent was removed in vacuo. The crude material was purified by flash chromatography (12:1 CH₂Cl₂:MeOH) yielding 93% product which is Formula II of the present disclosure (AH-003-193).

If Formula I is desired, going from FIG. 7—Step H, FIG. 7—Step I is performed. The deprotected dipeptide [from step H] in toluene was stirred for 5 days at 130° C. in pressure vessel. The reaction mixture was evaporated and then purified in chromatography using ethyl acetate/hexane system, yielding (70%) of Formula I (AH-004-199).

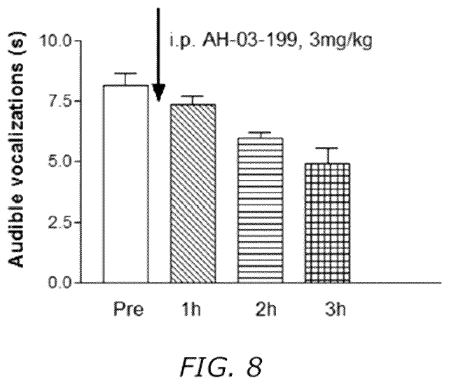

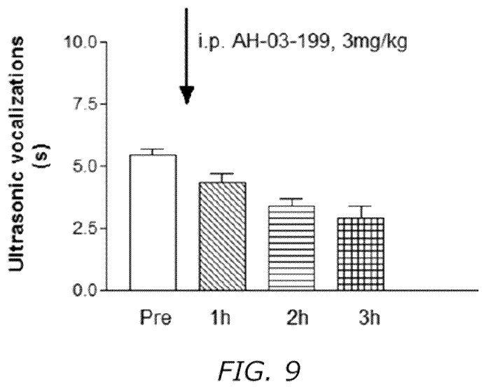

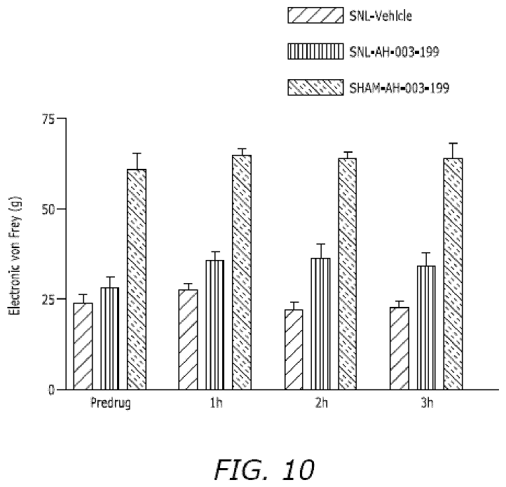

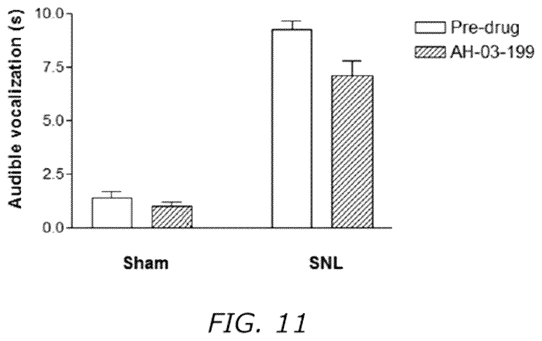

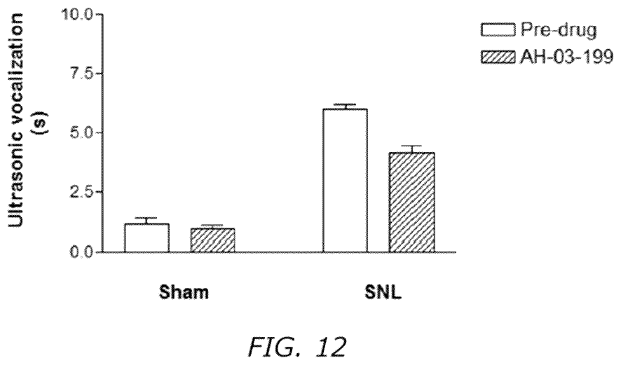

In another embodiment of the present invention, a compound of the Formula IV class was tested in an in vivo rodent study. FIGS. 8-12 provides data related to certain pain indices in rodent studies. FIG. 8 presents data relating to rodent-based audible vocalizations for Formula IV. FIG. 9 presents data relating to rodent-based ultrasonic vocalizations for Formula IV. FIG. 10 presents data relating to rodent-based Electronic von Frey measurements for Formula IV. FIG. 11 presents data relating to rodent-based audible vocalizations for Formula IV. FIG. 12 presents data relating to ultrasonic vocalizations for Formula IV. These studies have been considered to be valid, quantitative, reliable and convenient methods to measure pain-related behavior.

It is another embodiment of the present invention to provide an enantioselective synthesis method using Formulas I-IV, enhancing their pharmaceutical efficacy and specificity. This methodological embodiment can allow for the precise control over the stereochemistry of the synthesized compounds, ensuring that only the desired enantiomer is produced. This is particularly important in pharmaceutical applications, as different enantiomers of the same compound can have vastly different biological activities. By utilizing this enantioselective approach, the synthesized compounds can exhibit improved pharmacokinetic properties, reduced side effects, and increased therapeutic efficiency. Furthermore, this synthesis method could potentially streamline the manufacturing process, leading to more cost-effective production while maintaining high standards of drug purity and quality. Overall, the implementation of enantioselective synthesis represents a crucial step forward in the development and application of Formulas I-IV, offering a more targeted and effective approach to treatment.

It is another embodiment of the present invention to provide any of Formulas I-IV comprising an aqueous solution and one or more pharmaceutically acceptable excipients, additives, carriers or adjuvants. Any of Formulas I-IV may further comprise one or more excipients, carriers, additives, adjuvants, or binders in a tablet or capsule. Either Formula I or Formula II may further be administered via an oral, intraperitoneal, intravascular, peripheral circulation, subcutaneous, intraorbital, ophthalmic, intraspinal, intracisternal, topical, infusion, implant, aerosol, inhalation, scarification, intracapsular, intramuscular, intranasal, buccal, transdermal, pulmonary, rectal, or vaginal route.

The compounds of the present invention are capable of treatment in a manner selective to CNS activity and does not manipulate the activity of other CNS receptors, as other CNS drugs have a tendency to do. Therefore, the compounds of the present invention have substantially reduced toxicity profiles (i.e. depression, headache, suicidal thoughts, and the like). The compounds of the present invention are further active as low nanomolar ranges due to its potency.

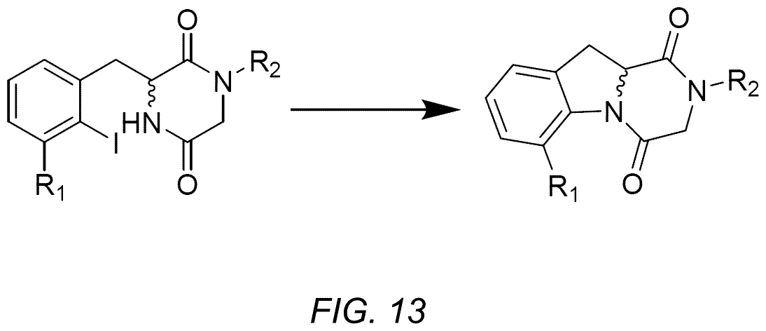

FIG. 13 depicts a synthesis schematic of Formula V of the present invention. This schematic outlines the step-by-step process involved in the synthesis of novel tricyclic diketopiperazine (DKP) analogs, designated as Formula V compounds. The synthesis can begin with the preparation of bicyclic DKP intermediates. These intermediates can be derived from corresponding dipeptide molecules, which may undergo intramolecular cyclization under basic conditions, specifically using ammonium hydroxide and an Arrhenius base. This initial stage can establish the bicyclic framework that may be pivotal for the subsequent development of the tricyclic system.

The synthesis process may advance to the final step where the tricyclic DKP-based compounds can be synthesized. In some embodiments, the final step is achieved through an Ullmann cross-coupling reaction, as depicted in FIG. 13. The Ullmann reaction, in such an embodiment, introduces various substituents at specific positions on the tricyclic DKP scaffold, leading to the formation of diverse analogs with distinct chemical properties and biological activities.

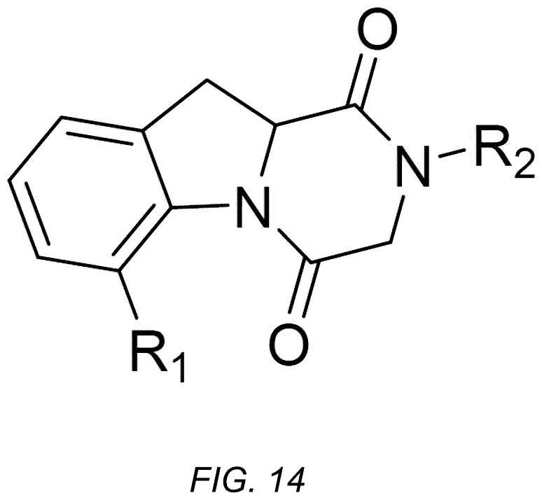

FIG. 14, in turn, depicts the chemical structure of Formula V, illustrating the tricyclic system containing the DKP moiety. FIG. 14 provides the molecular architecture of the compound. In some embodiments of Formula V, the compound may feature a methoxy group at the R1 position and a methyl group at the R2 position. In other embodiments, the R1 position can hold a methoxy group while the R2 position may be occupied by a hydrogen atom. Further variations can include a methoxy group at R1 paired with a benzyl group at R2, and another embodiment may feature a hydrogen atom at R1 and a methyl group at R2. These variations in substituents can be crucial as they may contribute to the compound's affinity and specificity towards opioid receptors.

In one embodiment, R1 is a chemical group selected from a group consisting of: hydrogen, alkyl, hydroxy, alkyloxy, alkyl ester, amine, alkylamine, dialkylamine, thio, thioalkyl, and alkyl.

In one embodiment, R2 is a chemical group selected from a group consisting of: hydrogen, benzyl, substituted benzyl, methyl, alkyl carbamate, alkyl, prenyl, and cycloalkyl.

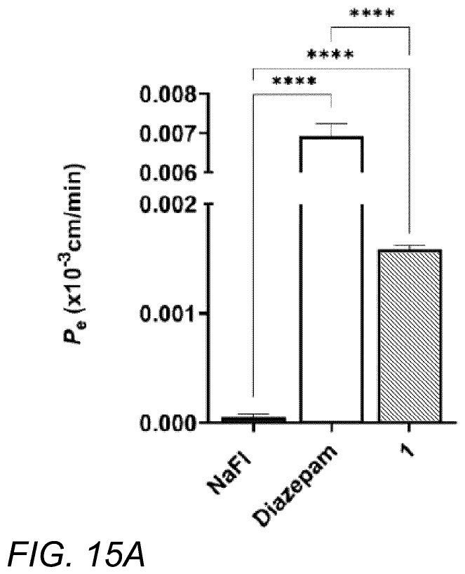

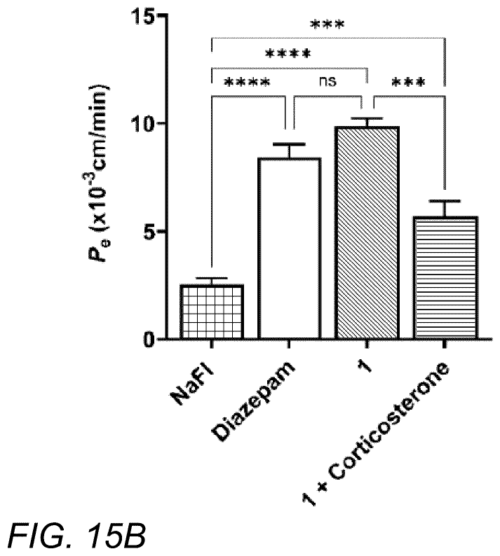

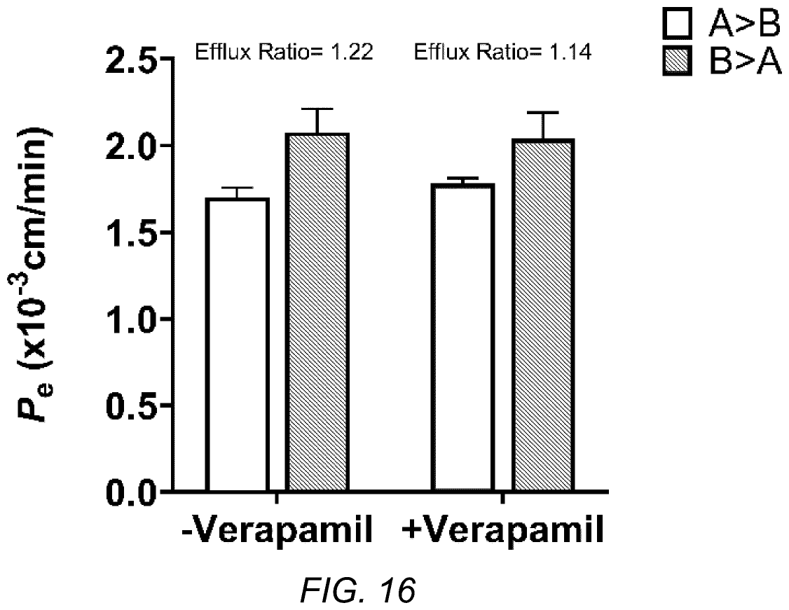

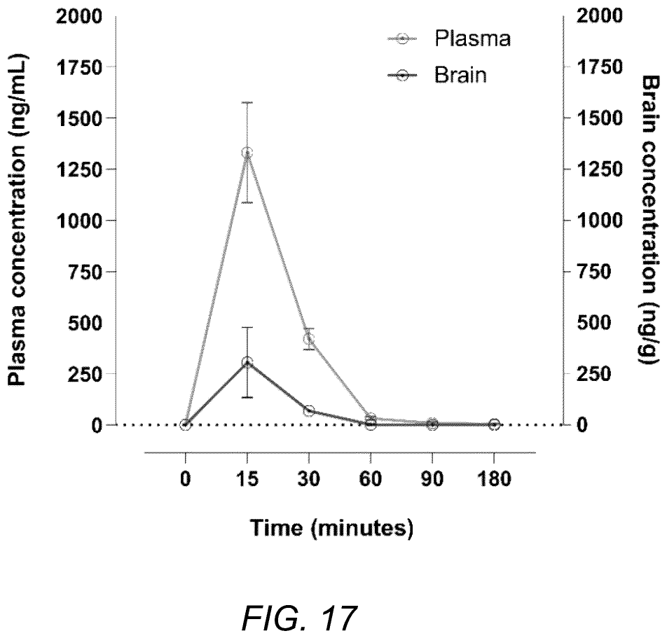

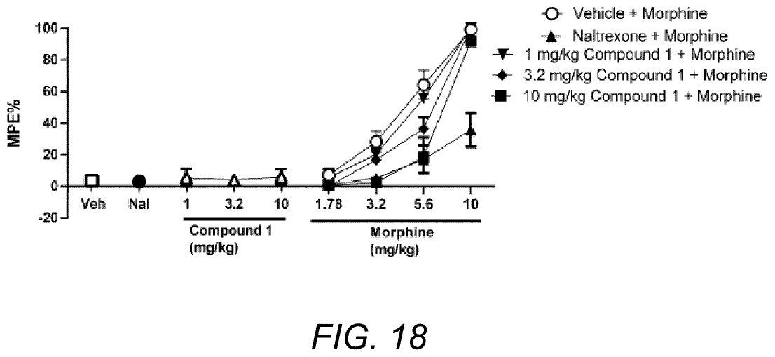

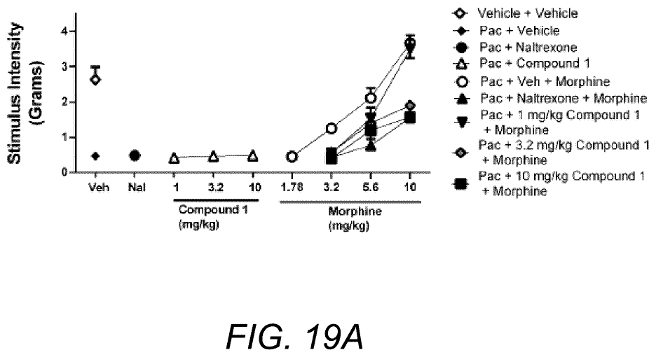

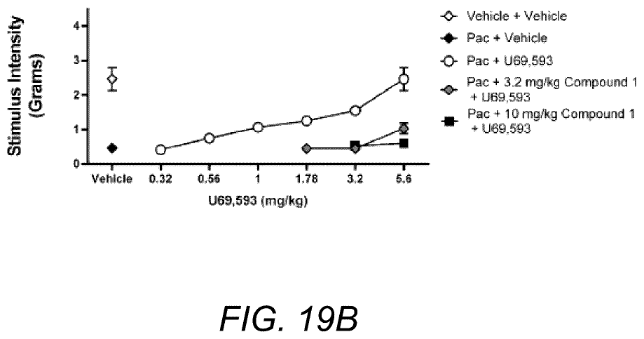

In another embodiment of the present invention, a compound of the Formula V class was tested in an in vivo rodent study. FIGS. 15A-19B provides data related to certain pain indices in rodent studies. FIG. 15A depicts data relating to basolateral transport in parallel artificial permeability assays for Formula V. FIG. 15B depicts data relating to basolateral transport across an in-vitro bEnd-3 monolayer model for Formula V. FIG. 16 depicts data relating to bidirectional transport in Caco-2 cell monolayers for Formula V. FIG. 17 depicts data relating to the rodent-based in-vivo pharmacokinetic profile of Formula V. FIG. 18 depicts data relating to the maximum possible antinociceptive effect of compounds, including Formula V, on rodents. FIG. 19A depicts data relating to the stimulus intensity of compounds, including Formula V, on paclitaxel-induced mechanical allodynia. FIG. 19B depicts data relating to the stimulus intensity of compounds, including Formula V, on paclitaxel-induced mechanical allodynia. These studies have been considered to be valid, quantitative, reliable and convenient methods to measure pain-related behavior.

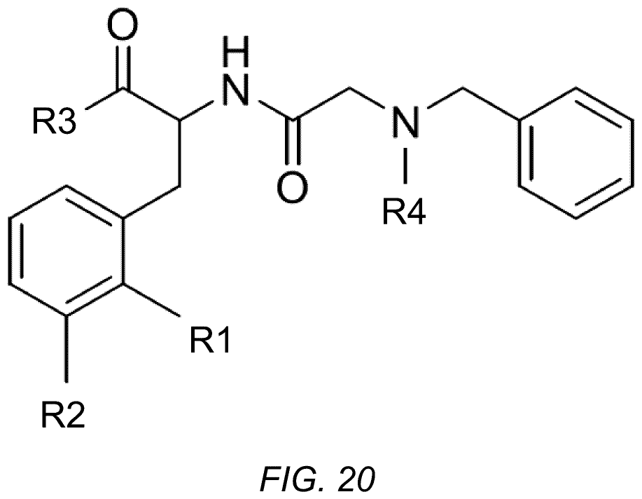

Turning to FIG. 20, Formula VI of the present invention is provided with the various R-groups. In one embodiment, R1 is a chemical group selected from a group consisting of: hydrogen, halogen, nitro, alkyl, aliphatic, cycloalkyl, trifluoroalkyl, substituted phenyl, carboxylic acid, alkyl ester of carboxylic acid, and acetyl.

In one embodiment, R2 is a chemical group selected from a group consisting of: hydrogen, hydroxy, alkyloxy, alkyl ester, amine, alkylamine, dialkylamine, thio, thioalkyl, and alkyl.

In one embodiment, R3 is a chemical group selected from a group consisting of: hydrogen, benzyl, substituted benzyl, methyl, alkyl carbamate, alkyl, prenyl, dialkylamide, and cycloalkyl.

In one embodiment, R4 is a chemical group selected from a group consisting of: hydrogen, alkyl, acetyl, cycloalkyl, benzyl, and substituted benzyl.

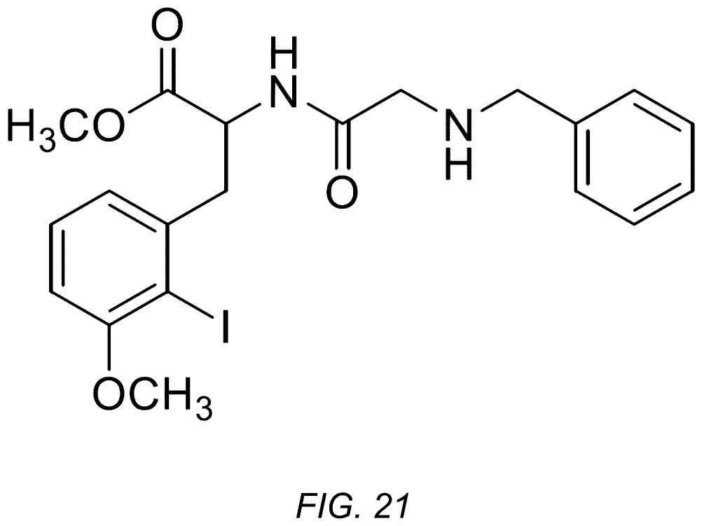

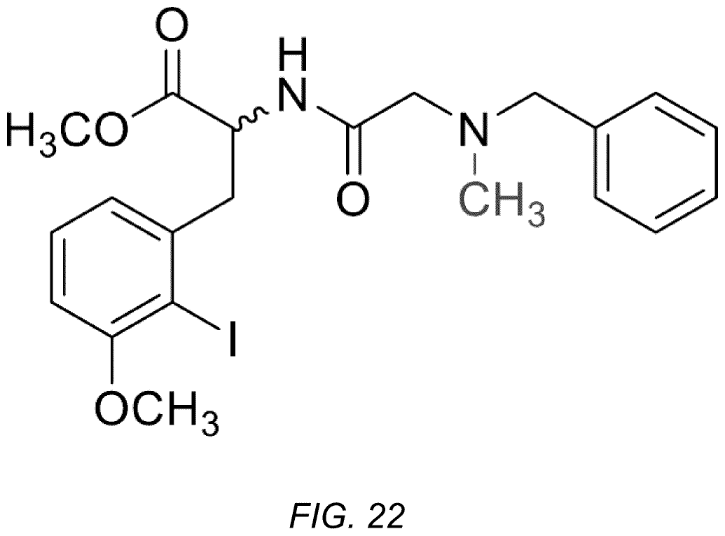

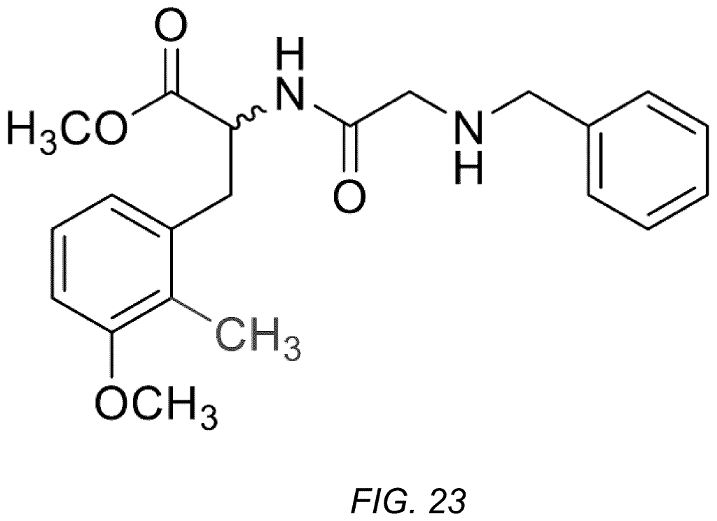

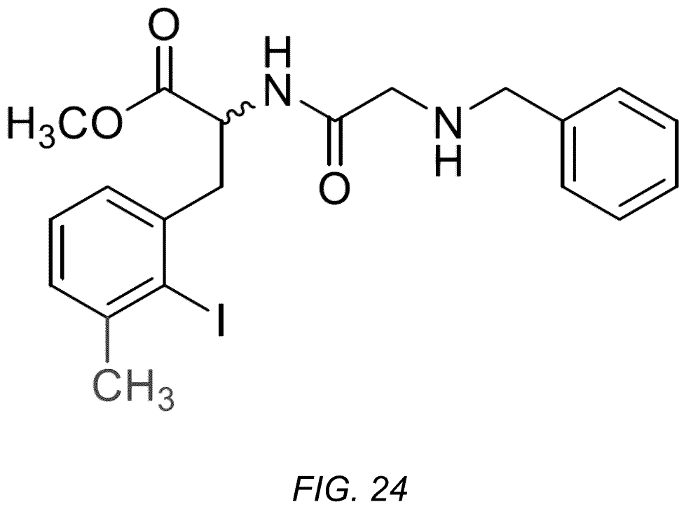

Turning to FIG. 21, Formula VII of the present invention is provided. The inhibition of the KOR is quantified in Table 1, as is the inhibition constant (Ki) value. FIG. 22 presents Formula VIII of the present invention. The inhibition of the KOR is quantified in Table 1, as is the inhibition constant (Ki) value. FIG. 23 presents Formula IX of the present invention. The inhibition of the KOR is quantified in Table 1, as is the inhibition constant (Ki) value. FIG. 24 presents Formula X of the present invention. The inhibition of the KOR is quantified in Table 1, as is the inhibition constant (Ki) value.

Formula VII, Formula VIII, Formula IX, and Formula X are capable of maintaining KOR antagonist properties in the nanomolar range.

It is therefore an embodiment of the present invention to provide novel compounds of Formula VII, Formula VIII, Formula IX, and Formula X, and derivatives thereof, or pharmaceutically acceptable salts thereof, which are presented for treating certain disorders in a patient, such as addiction, alcohol dependence, opioid abuse treatment, neurological disorders, and neuropathic pain. It is another embodiment of the present invention to provide pharmaceutically acceptable compositions comprising compounds of Formula VII, Formula VIII, Formula IX, and Formula X, and a pharmaceutically acceptable carrier.

Another embodiment of the present invention relates to a method for providing neuroprotection in a mammal in need of such treatment. The method comprises administering to the mammal a therapeutically effective amount of a compound of any of Formulas VI-X or a pharmaceutically acceptable salt thereof. In another embodiment, the present invention provides a pharmaceutical composition comprising a therapeutically effective amount of a compound of the invention or a pharmaceutically acceptable salt thereof in combination with one or more pharmaceutically acceptable carriers. The composition is preferably useful for the treatment of the disease conditions described above.

Further, the present invention provides the use of a compound of Formulas VI-X or a pharmaceutically acceptable salt thereof, in the manufacture of a medicament for the treatment of the disease conditions described above.

In another embodiment, the compounds, compositions, and methods disclosed herein therefore may be utilized to prevent and/or treat a disease such as alcohol dependence, opioid abuse treatment, neurological disorders, neuropathic pain, and fibromyalgia, among other conditions mediated by one or more opioid receptors.

In an illustrative embodiment of the present invention, Formula VII, Formula VIII, Formula IX, and Formula X, were evaluated for selective activity of the KOR. Turning to Table 3, inhibitory data is provided for the KOR. Inhibition data is provided as inhibitor constant, Ki, is the concentration required to produce half-maximum inhibition. This quantitative measure indicates how much of a particular drug or other substance (inhibitor) is needed to inhibit a given biological process (or component of a process, i.e. an enzyme, cell, cell receptor or microorganism) by half. The values are typically expressed as molar (micro- and nano-) concentrations.

TABLE 3
Inhibitory data relating to KOR inhibition of Formula VII, Formula VIII,
Formula IX, and Formula X of the present invention.

| | Formula VII | Formula VIII | Formula IX | Formula X |
|---|---|---|---|---|
| Ki | 277 nM | 256 nM | 192 nM | 122 nM |

In one embodiment, compound of the present invention either Formula VII, Formula VIII, Formula IX, and Formula X, are shown as in Table 3 to show inhibition with regard to various receptors, including opioid receptors μ (MOR), σ (DOR), and κ (KOR).

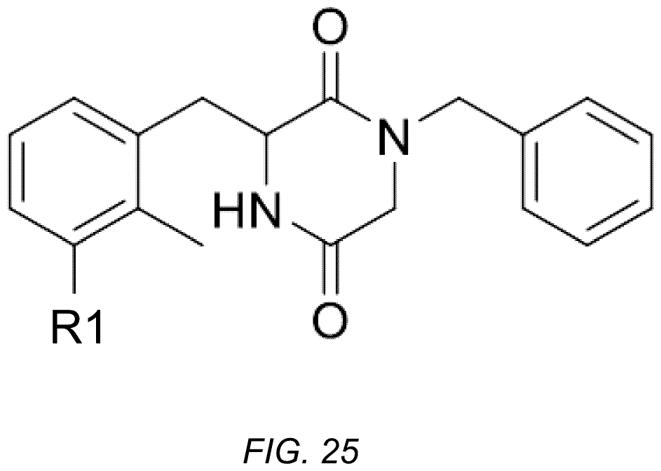

Turning to FIG. 25, Formula XI of the present invention is provided with the various R-groups. In one embodiment, R1 is a chemical group selected from a group consisting of: hydrogen, halogen, nitro, alkyl, aliphatic, cycloalkyl, trifluoroalkyl, substituted phenyl, carboxylic acid, alkyl ester of carboxylic acid, and acetyl.

It is therefore an embodiment of the present invention to provide novel compounds of XI, and derivatives thereof, or pharmaceutically acceptable salts thereof, which are presented for treating certain disorders in a patient, such as addiction, alcohol dependence, opioid abuse treatment, neurological disorders, and neuropathic pain. It is another embodiment of the present invention to provide pharmaceutically acceptable compositions comprising compounds of Formula XI, and a pharmaceutically acceptable carrier.

Another embodiment of the present invention relates to a method for providing neuroprotection in a mammal in need of such treatment. The method comprises administering to the mammal a therapeutically effective amount of a compound of any of Formula XI or a pharmaceutically acceptable salt thereof. In another embodiment, the present invention provides a pharmaceutical composition comprising a therapeutically effective amount of a compound of the invention or a pharmaceutically acceptable salt thereof in combination with one or more pharmaceutically acceptable carriers. The composition is preferably useful for the treatment of the disease conditions described above.

Further, the present invention provides the use of a compound of Formula XI or a pharmaceutically acceptable salt thereof, in the manufacture of a medicament for the treatment of the disease conditions described above.

In another embodiment, the compounds, compositions, and methods disclosed herein therefore may be utilized to prevent and/or treat a disease such as alcohol dependence, opioid abuse treatment, neurological disorders, neuropathic pain, and fibromyalgia, among other conditions mediated by one or more opioid receptors.

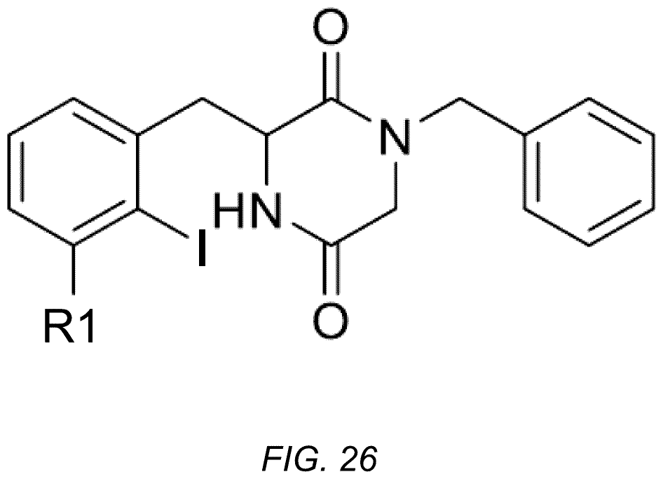

Turning to FIG. 26, Formula XII of the present invention is provided with the various R-groups. In one embodiment, R1 is a chemical group selected from a group consisting of: hydrogen, halogen, nitro, alkyl, aliphatic, cycloalkyl, trifluoroalkyl, substituted phenyl, carboxylic acid, alkyl ester of carboxylic acid, and acetyl.

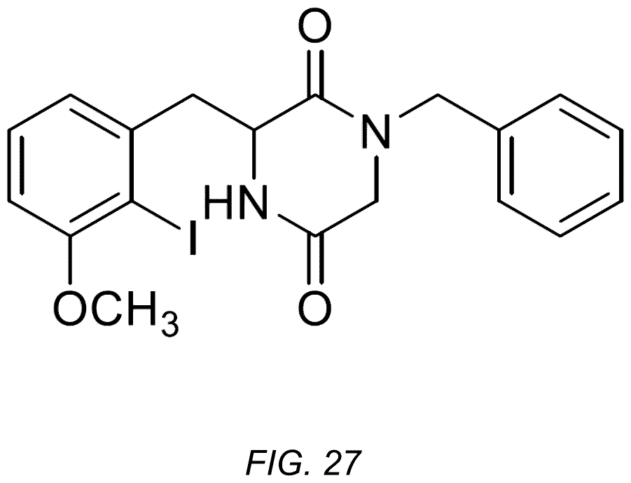

Turning to FIG. 27, Formula XIII of the present invention is provided. The inhibition of the KOR is quantified in Table 1, as is the inhibition constant (Ki) value. Formula XIII is capable of maintaining KOR antagonist properties in the nanomolar range.

It is therefore an embodiment of the present invention to provide novel compounds of Formula XIII, and derivatives thereof, or pharmaceutically acceptable salts thereof, which are presented for treating certain disorders in a patient, such as addiction, alcohol dependence, opioid abuse treatment, neurological disorders, and neuropathic pain. It is another embodiment of the present invention to provide pharmaceutically acceptable compositions comprising compounds of Formula XIII, and a pharmaceutically acceptable carrier.

Another embodiment of the present invention relates to a method for providing neuroprotection in a mammal in need of such treatment. The method comprises administering to the mammal a therapeutically effective amount of a compound of any of Formula XIII or a pharmaceutically acceptable salt thereof. In another embodiment, the present invention provides a pharmaceutical composition comprising a therapeutically effective amount of a compound of the invention or a pharmaceutically acceptable salt thereof in combination with one or more pharmaceutically acceptable carriers. The composition is preferably useful for the treatment of the disease conditions described above.

Further, the present invention provides the use of a compound of Formula XIII or a pharmaceutically acceptable salt thereof, in the manufacture of a medicament for the treatment of the disease conditions described above.

In another embodiment, the compounds, compositions, and methods disclosed herein therefore may be utilized to prevent and/or treat a disease such as alcohol dependence, opioid abuse treatment, neurological disorders, neuropathic pain, and fibromyalgia, among other conditions mediated by one or more opioid receptors.

In an illustrative embodiment of the present invention, Formula XIII was evaluated for selective activity of the KOR. Turning to Table 4, inhibitory data is provided for the KOR. Inhibition data is provided as inhibitor constant, Ki, is the concentration required to produce half-maximum inhibition. This quantitative measure indicates how much of a particular drug or other substance (inhibitor) is needed to inhibit a given biological process (or component of a process, i.e. an enzyme, cell, cell receptor or microorganism) by half. The values are typically expressed as molar (micro- and nano-) concentrations. The data in Table 4 was obtained for the racemic mixture of Formula XIII. In some embodiments, the specific enantiomer of this compound demonstrates a Ki of 425 nM, indicating a significant level of selectivity and potency.

TABLE 4
Inhibitory data relating to KOR inhibition of Formula I and
Formula II of the present invention.

| | Formula XIIII |
|---|---|
| Ki | 924 nM |
| Inhibition (%) | 74.5% |

In one embodiment, compound of the present invention Formula XIII is shown as in Table 4 to show inhibition with regard to various receptors, including opioid receptors (MOR), a (DOR), and x (KOR).

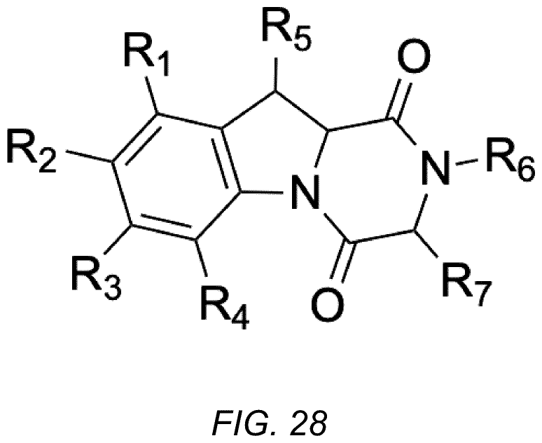

Turning to FIG. 28, Formula XIV of the present invention is provided with the various R-groups. In one embodiment, R1 is a chemical group selected from a group consisting of: hydrogen, halogen, amine, alkyl amine, alkyl, hydroxy-, alkylhydroxy, alkyl amide, and alkyl ester.

In one embodiment, R2 is a chemical group selected from a group consisting of: hydrogen, halogen, amine, alkyl amine, alkyl, hydroxy-, alkylhydroxy, alkyl amide, and alkyl ester.

In one embodiment, R3 is a chemical group selected from a group consisting of: hydrogen, halogen, amine, alkyl amine, alkyl, hydroxy-, alkylhydroxy, alkyl amide, and alkyl ester.

In one embodiment, R4 is a chemical group selected from a group consisting of: hydrogen, halogen, amine, alkyl amine, alkyl, hydroxy-, alkylhydroxy, alkyloxy, alkyl amide, and alkyl ester.

In one embodiment, R5 is a chemical group selected from a group consisting of: hydrogen, halogen, amine, alkyl, alkyl amine, hydroxy-, alkylhydroxy-, alkyl amide, alkyl ester, benzyl, and phenyl alkyl.

In one embodiment, R6 is a chemical group selected from a group consisting of: hydrogen, halogen, amine, alkyl, alkyl amine, hydroxy-, alkylhydroxy-, alkyl amide, and alkyl ester.

In one embodiment, R7 is a chemical group selected from a group consisting of: hydrogen, halogen, amine, alkyl, alkyl amine, hydroxy-, alkylhydroxy-, alkyl amide, alkyl ester, benzyl, and phenyl alkyl.

It is therefore an embodiment of the present invention to provide novel compounds of XIV, and derivatives thereof, or pharmaceutically acceptable salts thereof, which are presented for treating certain disorders in a patient, such as addiction, alcohol dependence, opioid abuse treatment, neurological disorders, and neuropathic pain. It is another embodiment of the present invention to provide pharmaceutically acceptable compositions comprising compounds of Formula XIV, and a pharmaceutically acceptable carrier.

Another embodiment of the present invention relates to a method for providing neuroprotection in a mammal in need of such treatment. The method comprises administering to the mammal a therapeutically effective amount of a compound of any of Formula XIV or a pharmaceutically acceptable salt thereof. In another embodiment, the present invention provides a pharmaceutical composition comprising a therapeutically effective amount of a compound of the invention or a pharmaceutically acceptable salt thereof in combination with one or more pharmaceutically acceptable carriers. The composition is preferably useful for the treatment of the disease conditions described above.

Further, the present invention provides the use of a compound of Formula XIV or a pharmaceutically acceptable salt thereof, in the manufacture of a medicament for the treatment of the disease conditions described above.

In another embodiment, the compounds, compositions, and methods disclosed herein therefore may be utilized to prevent and/or treat a disease such as alcohol dependence, opioid abuse treatment, neurological disorders, neuropathic pain, and fibromyalgia, among other conditions mediated by one or more opioid receptors.

Those skilled in the art will recognize that the methods and compositions of the present invention may be implemented in many manners and as such are not to be limited by the foregoing exemplary embodiments and examples. In other words, functional elements being performed by single or multiple components, in various combinations of hardware and software or firmware, and individual functions, may be distributed among various software applications at either the client level or server level or both. In this regard, any number of the features of the different embodiments described herein may be combined into single or multiple embodiments, and alternate embodiments having fewer than, or more than, all of the features described herein are possible.

Functionality may also be, in whole or in part, distributed among multiple components, in manners now known or to become known. Thus, myriad combinations are possible in achieving the functions, features, and preferences described herein. Moreover, the scope of the present invention covers conventionally known manners for carrying out the described features as well as those variations and modifications that may be made to the processes, composition, or compounds described herein as would be understood by those skilled in the art now and hereafter.

While various embodiments have been described for purposes of this disclosure, such embodiments should not be deemed to limit the teaching of this disclosure to those embodiments. Various changes and modifications may be made to the elements and operations described above to obtain a result that remains within the scope of the compositions and methods described in this disclosure.

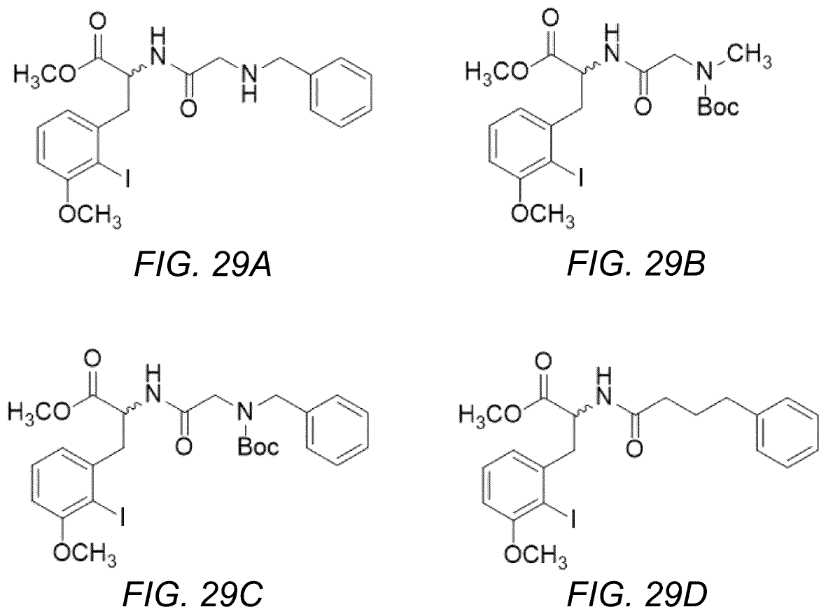

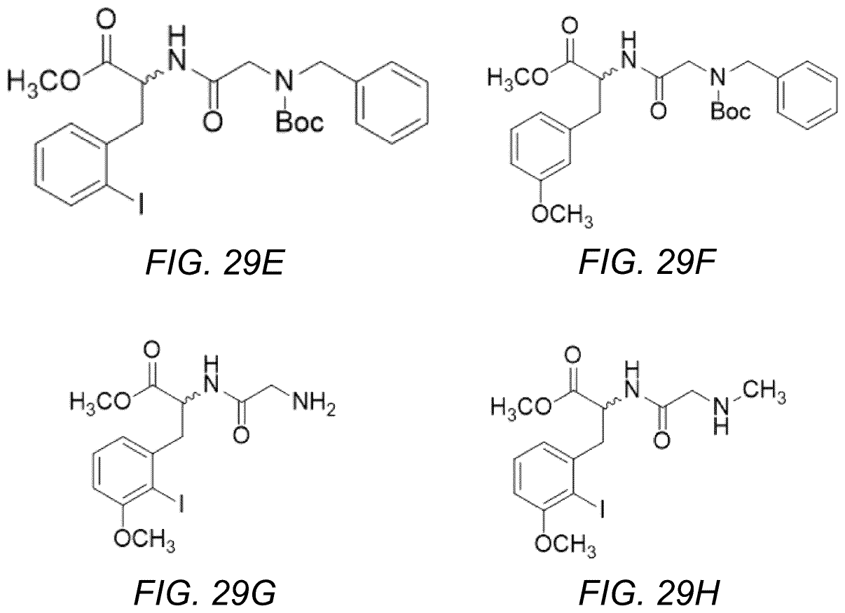

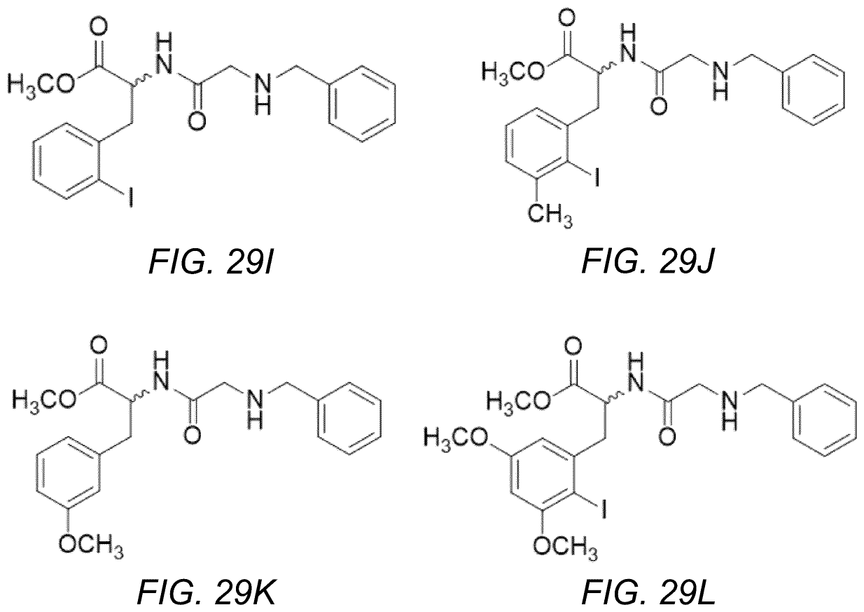

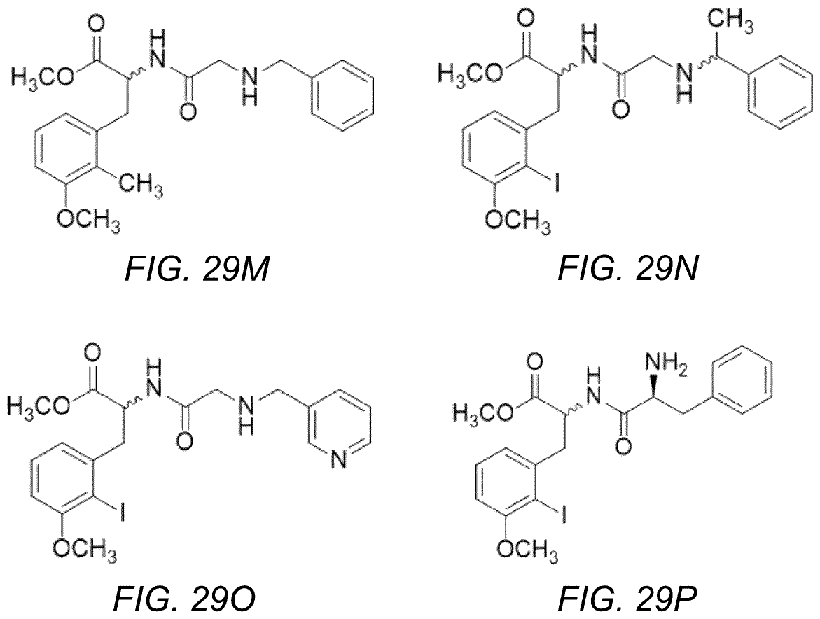

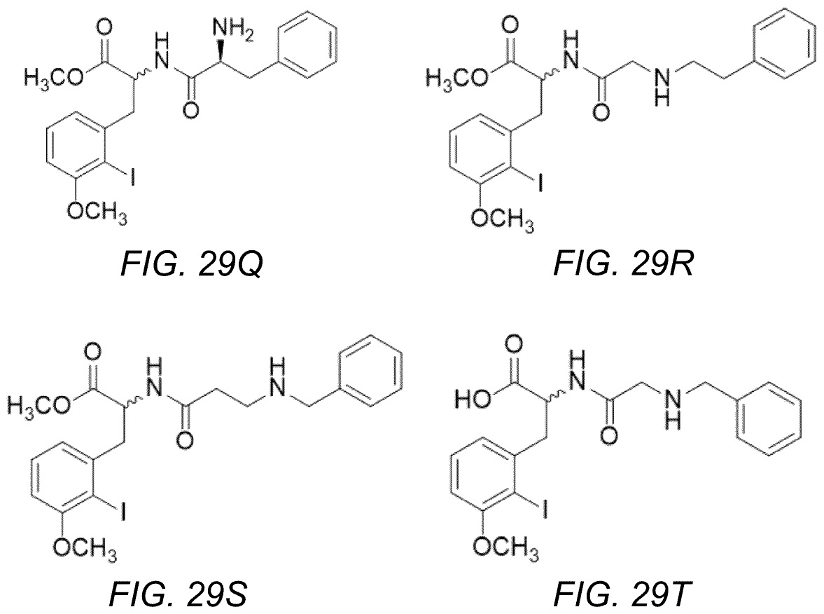

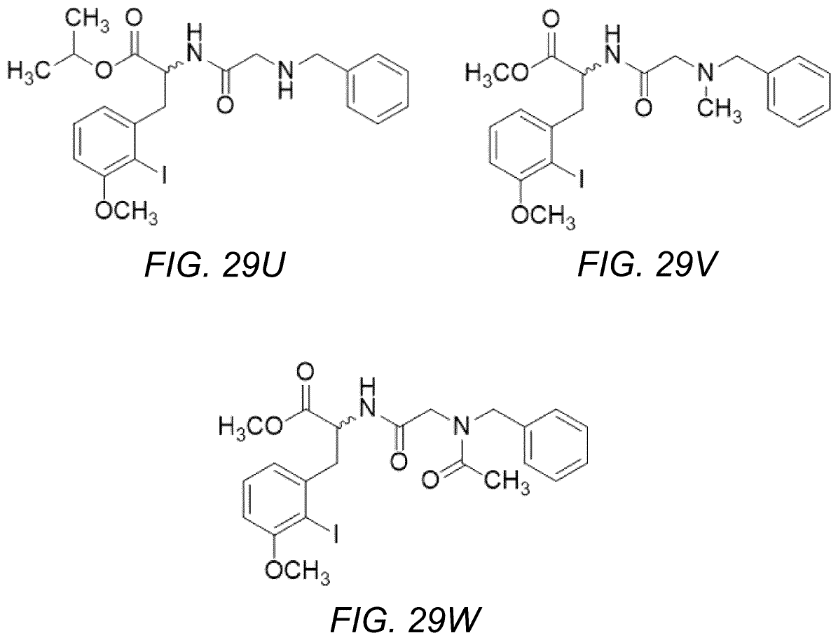

FIGS. 29A-29W depict a series of “open chain” analogs. FIG. 29A depicts the chemical structure for Formula XV. FIG. 29B depicts the chemical structure for Formula XVI. FIG. 29C depicts the chemical structure for Formula XVII. FIG. 29D depicts the chemical structure for Formula XVIII. FIG. 29E depicts the chemical structure for Formula XIX. FIG. 29F depicts the chemical structure for Formula XX. FIG. 29G depicts the chemical structure for Formula XXI. FIG. 29H depicts the chemical structure for Formula XXII. FIG. 29I depicts the chemical structure for Formula XXIII. FIG. 29J depicts the chemical structure for Formula XXIV. FIG. 29K depicts the chemical structure for Formula XXV. FIG. 29L depicts the chemical structure for Formula XXVI. FIG. 29M depicts the chemical structure for Formula XXVII. FIG. 29N depicts the chemical structure for Formula XXVIII. FIG. 29O depicts the chemical structure for Formula XXIX. FIG. 29P depicts the chemical structure for Formula XXX. FIG. 29Q depicts the chemical structure for Formula XXXI. FIG. 29R depicts the chemical structure for Formula XXXII. FIG. 29S depicts the chemical structure for Formula XXXIII. FIG. 29T depicts the chemical structure for Formula XXXIV. FIG. 29U depicts the chemical structure for Formula XXXV. FIG. 29V depicts the chemical structure for Formula XXXVI. FIG. 29W depicts the chemical structure for Formula XXXVII.

In certain embodiments of the present invention, the series of “open chain” analogs of Formulas XV to XXXVII, as depicted in FIGS. 29A-29W, respectively, can include compounds that vary in their structural configurations. These analogs can be characterized by their unique chemical structures, ranging from Formula XV to Formula XXXVII. In some embodiments, the analogs can interact with the Kappa Opioid Receptor (KOR) in various manners, influenced by the structural elements present in each compound.

In some embodiments, the “open chain” analogs of Formulas XV to XXXVII, as depicted in FIGS. 29A-29W, respectively, can exhibit different binding activities to the KOR. For example, the presence of certain structural elements such as an alkyl linker or an electron-deficient analog can impact the compound's interaction with the receptor. In certain embodiments, modifications in the structure of these analogs can lead to variations in their binding efficacy to KOR. Additionally, the role of specific functionalities, such as amine groups, can be pivotal in certain embodiments, with their hydrogen-bond acceptor characteristics playing a significant role.

In various embodiments, the “open chain” analogs of Formulas XV to XXXVII, as depicted in FIGS. 29A-29W, respectively, can demonstrate a range of pharmacological effects. These effects can include, but are not limited to, the potential for blood-brain barrier permeability and metabolic stability. For example, certain compounds within this series can exhibit higher permeability compared to standard controls without affecting the integrity of the blood-brain barrier. Furthermore, in some embodiments, the metabolic stability of these compounds can vary across different species, with potential implications for their pharmacokinetic profiles.

In certain embodiments, the “open chain” analogs of Formulas XV to XXXVII, as depicted in FIGS. 29A-29W, respectively, can have therapeutic potential in the management of pain. For instance, specific compounds within this series can reduce behavioral symptoms associated with neuropathic pain, as observed in established animal models. In some embodiments, the efficacy of these compounds can be comparable to standard treatments but may require lower dosages, indicating their potential advantage in clinical settings.

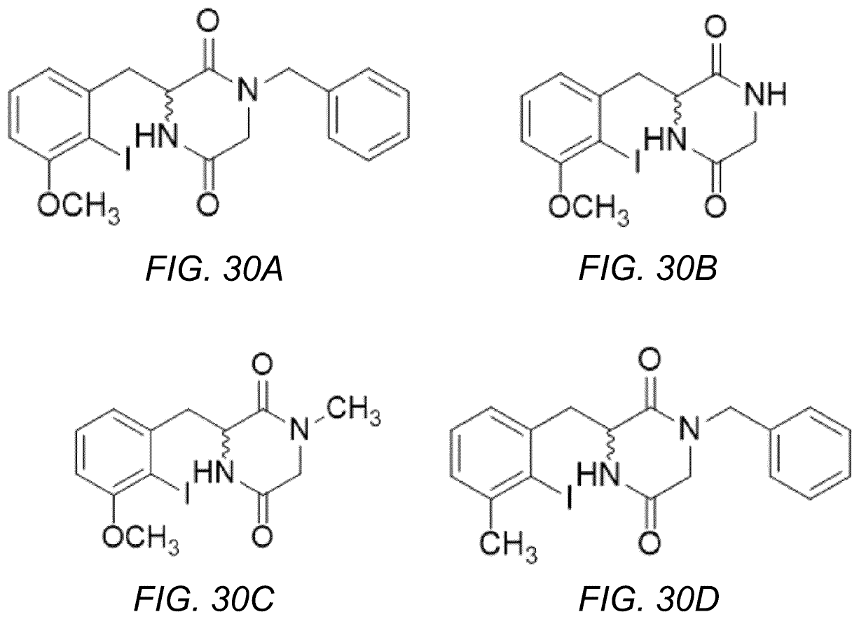

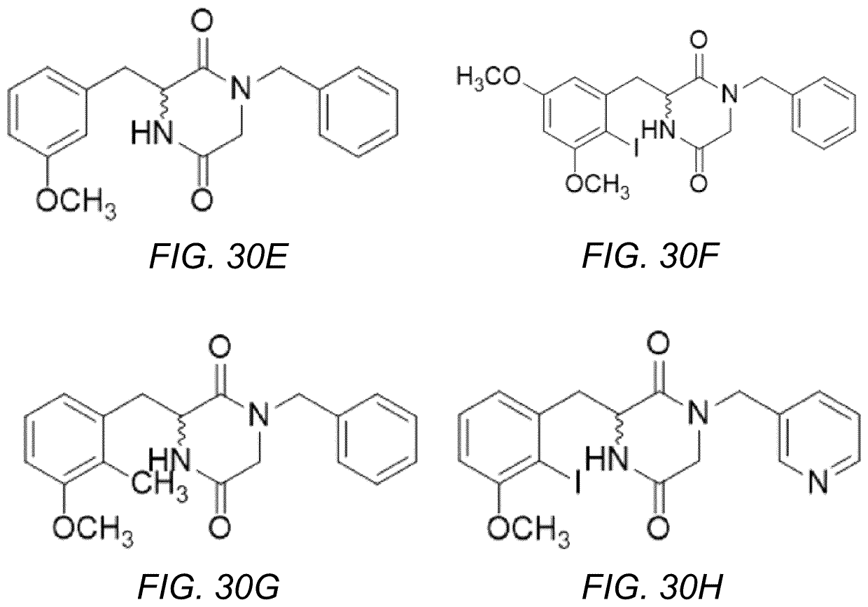

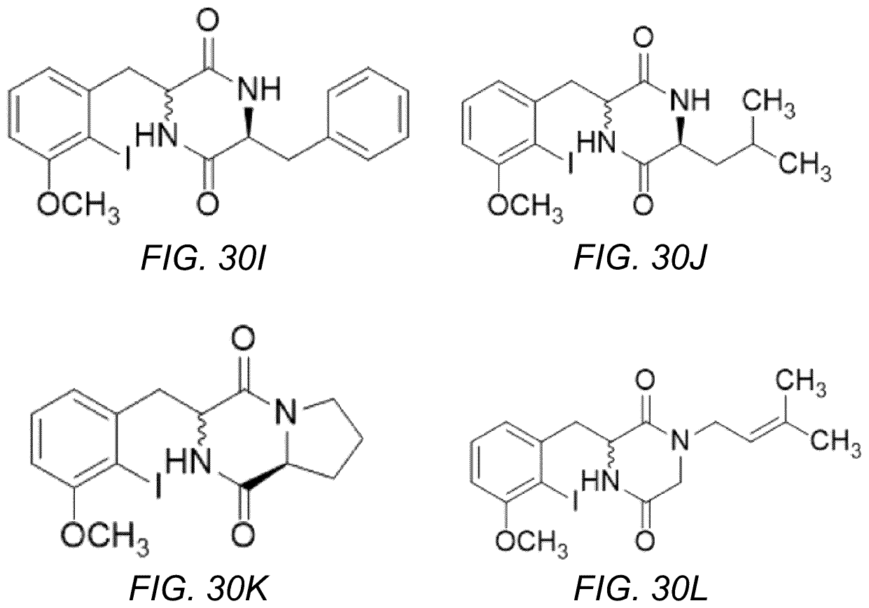

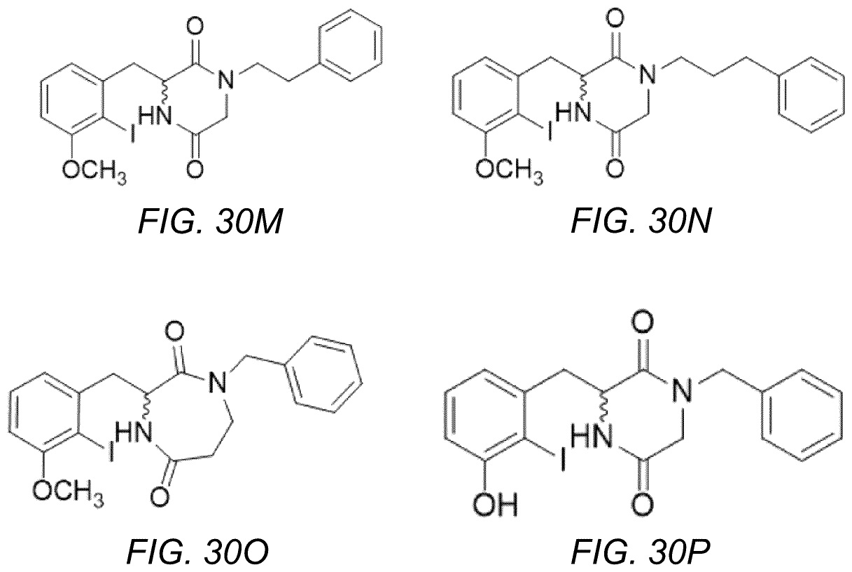

FIGS. 30A-30P depict a series of “closed chain” analogs. FIG. 30A depicts the chemical structure for Formula XXXVIII. FIG. 30B depicts the chemical structure for Formula XXXIX. FIG. 30C depicts the chemical structure for Formula XL. FIG. 30D depicts the chemical structure for Formula XLI. FIG. 30E depicts the chemical structure for Formula XLII. FIG. 30F depicts the chemical structure for Formula XLIII. FIG. 30G depicts the chemical structure for Formula XLIV. FIG. 30H depicts the chemical structure for Formula XLV. FIG. 30I depicts the chemical structure for Formula XLVI. FIG. 30J depicts the chemical structure for Formula XLVII. FIG. 30K depicts the chemical structure for Formula XLVIII. FIG. 30L depicts the chemical structure for Formula XLIX. FIG. 30M depicts the chemical structure for Formula L. FIG. 30N depicts the chemical structure for Formula LI. FIG. 30O depicts the chemical structure for Formula LII. FIG. 30P depicts the chemical structure for Formula LIII.

In certain embodiments of the present invention, FIGS. 30A-30P depict a series of “closed chain” analogs, each illustrated by their respective chemical structures, ranging from Formula XXXVIII to LIII. These analogs can exhibit diverse structural configurations and can be characterized by variations in their chemical compositions.

In some embodiments, the “closed chain” analogs of Formulas XXXVIII to LIII, as depicted in FIGS. 30A-30P, respectively, can demonstrate varying binding affinities to the KOR. For example, certain compounds within this series can exhibit higher affinity compared to original hit molecules, as indicated by their Ki values. In certain embodiments, the role of hydrogen bond acceptors (HBA), but not hydrogen bond donors (HBD), can be critical in parts of these molecules. Additionally, the presence of specific substituents and alterations in the structure, such as the introduction of an additional methoxy group or changes in DKP substituents, can significantly influence the binding profiles and affinities of these analogs.

In various embodiments, the “closed chain” analogs of Formulas XXXVIII to LIII, as depicted in FIGS. 30A-30P, respectively, can have important pharmacological implications. For instance, certain modifications in these compounds can lead to increased or decreased binding affinities, impacting their potential therapeutic applications. In some embodiments, the compounds can show no toxicity against specific cell lines and demonstrate the ability to penetrate the blood-brain barrier, as assessed through in vitro studies. Furthermore, the plasma stability of these compounds can vary across different species, with some showing enhanced stability and others being rapidly metabolized, as observed in pharmacokinetic studies.

In certain embodiments, the structure-activity relationship analysis of these “closed chain” analogs of Formulas XXXVIII to LIII, as depicted in FIGS. 30A-30P, respectively, can provide insights into their interaction with KOR and potential for therapeutic use. For example, the analysis might suggest that carboxylic acid analogs have no affinity to KOR, guiding future studies to focus on improving stability and binding affinity using bioisostere strategies. Additionally, these compounds can be selective for KOR, showing minimal activity at other opioid receptors.

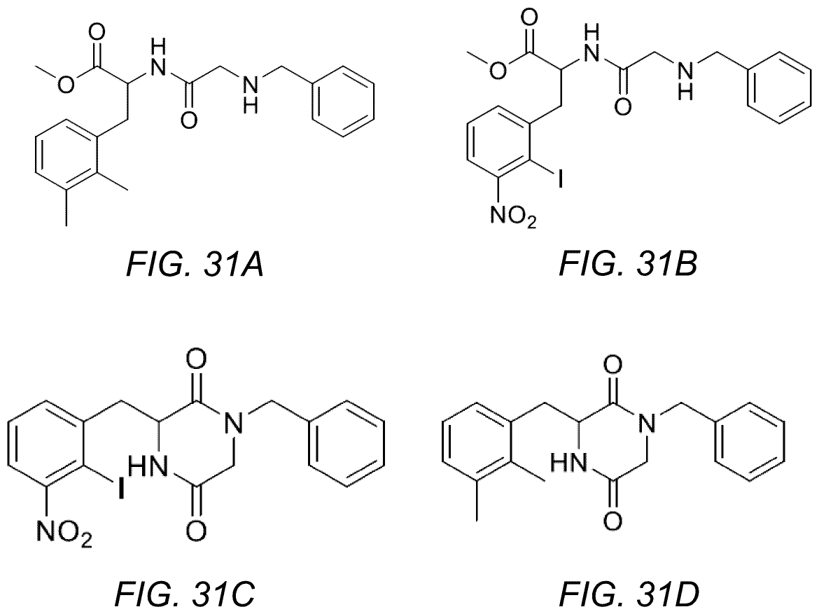

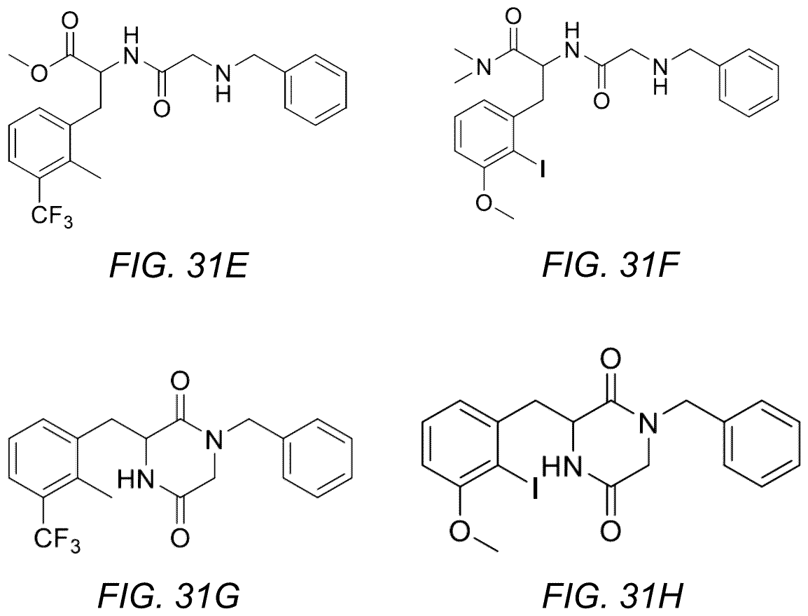

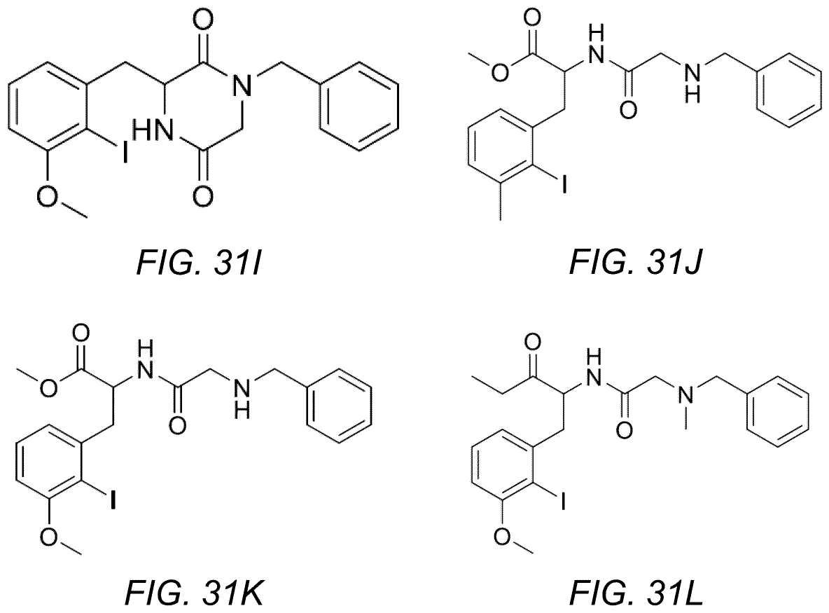

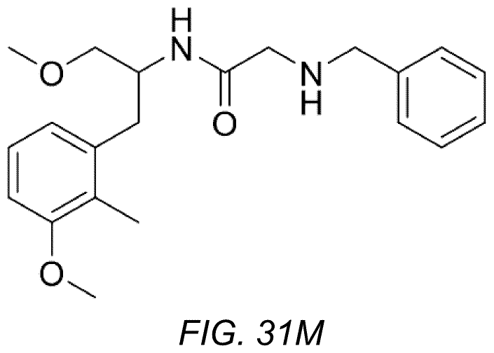

FIGS. 31A-31M depict a series of exemplary embodiments of analogs that are structured based on the chemical structure of Formula VI. FIG. 31A depicts the chemical structure for Formula LIV. FIG. 31B depicts the chemical structure for Formula LV. FIG. 31C depicts the chemical structure for Formula LVI. FIG. 31D depicts the chemical structure for Formula LVII. FIG. 31E depicts the chemical structure for Formula LVIII. FIG. 31F depicts the chemical structure for Formula LIX. FIG. 31G depicts the chemical structure for Formula LX. FIG. 31H depicts the chemical structure for Formula LXI. FIG. 31I depicts the chemical structure for Formula LXII. FIG. 31J depicts the chemical structure for Formula LXIII. FIG. 31K depicts the chemical structure for Formula LXIV. FIG. 31L depicts the chemical structure for Formula LXV. FIG. 31M depicts the chemical structure for Formula LXVI.

The foregoing description, for purposes of explanation, used specific nomenclature to provide a thorough understanding of the described embodiments. However, it should be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclosed. It should be apparent to one of ordinary skill in the art that many modifications and variations are possible in view of the above teachings.

While embodiments of the disclosure have been shown and described, modifications thereof can be made by one skilled in the art without departing from the spirit and teachings of the disclosure. The embodiments described and the examples provided herein are exemplary only, and are not intended to be limiting. Many variations and modifications of the disclosure disclosed herein are possible and are within the scope of the disclosure. The scope of protection is not limited by the description set out above, but is only limited by the claims which follow, that scope including all equivalents of the subject matter of the claims. Embodiments can include a system, a method, a compound, or combinations thereof.

What is claimed is:

1. A compound having a formula: 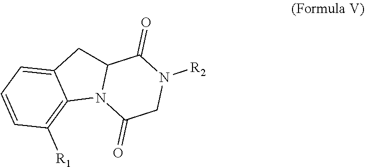
or a pharmaceutically acceptable salt thereof or isotopic variants thereof, stereoisomers or tautomers thereof.

2. The compound of claim 1, wherein R1 is a chemical group selected from a group consisting of: hydrogen, halogen, nitro, alkyl, alkyloxy, aliphatic, cycloalkyl, trifluoroalkyl, substituted phenyl, carboxylic acid, alkyl ester of carboxylic acid, and acetyl.

3. The compound of claim 1, wherein R2 is a chemical group selected from a group consisting of: hydrogen, hydroxy, alkyloxy, alkyl ester, amine, alkylamine, dialkylamine, thio, thioalkyl, and alkyl ethers.

4. A pharmaceutical composition comprising a therapeutically effective amount of the compound of claim 1, or a pharmaceutically acceptable salt, isotopic variants, stereoisomers or tautomers thereof.

5. A pharmaceutical formulation comprising an effective amount of a compound of claim 1 sufficient as a non-selective antagonist of opioid receptors.

6. The compound of claim 1, wherein said compound is effective to treat alcohol dependence, opioid abuse treatment, neurological disorders, neuropathic pain, and fibromyalgia.

7. The compound of claim 1, wherein said compound is further effective to block or reduce the tolerance of said human to an opioid receptor agonist.

8. A compound having a formula: 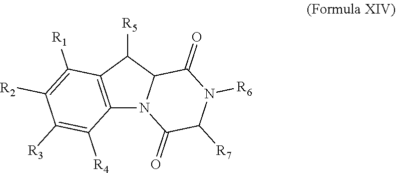
or a pharmaceutically acceptable salt thereof or isotopic variants thereof, stereoisomers or tautomers thereof.

9. The compound of claim 8, wherein R1 is a chemical group selected from a group consisting of: hydrogen, halogen, amine, alkyl amine, alkyl, hydroxy-, alkylhydroxy, alkyloxy, alkyl amide, and alkyl ester.

10. The compound of claim 8, wherein R2 is a chemical group selected from a group consisting of: hydrogen, halogen, amine, alkyl amine, alkyl, hydroxy-, alkylhydroxy, alkyl amide, and alkyl ester.

11. The compound of claim 8, wherein R3 is a chemical group selected from a group consisting of: hydrogen, halogen, amine, alkyl amine, alkyl, hydroxy-, alkylhydroxy, alkyl amide, and alkyl ester.

12. The compound of claim 8, wherein R4 is a chemical group selected from a group consisting of: hydrogen, halogen, amine, alkyl amine, alkyl, hydroxy-, alkylhydroxy, alkyl amide, and alkyl ester.

13. The compound of claim 8, wherein R5 is a chemical group selected from a group consisting of: hydrogen, halogen, amine, alkyl, alkyl amine, hydroxy-, alkylhydroxy-, alkyl amide, alkyl ester, benzyl, and phenyl alkyl.

14. The compound of claim 8, wherein R6 is a chemical group selected from a group consisting of: hydrogen, halogen, amine, alkyl, alkyl amine, hydroxy-, alkylhydroxy-, alkyl amide, and alkyl ester.

15. The compound of claim 8, wherein R7 is a chemical group selected from a group consisting of: hydrogen, halogen, amine, alkyl, alkyl amine, hydroxy-, alkylhydroxy-, alkyl amide, alkyl ester, benzyl, and phenyl alkyl.

16. The compound of claim 8, comprising a pharmaceutical composition having a therapeutically effective amount of said compound or a pharmaceutically acceptable salt thereof or isotopic variants thereof, stereoisomers or tautomers thereof.

17. The compound of claim 8, wherein said compound is capable of having at least 50% of the administered amount cross the blood-brain barrier (BBB) of a patient.

18. The compound of claim 8, wherein said compound is effective to treat addiction, alcohol dependence, opioid abuse treatment, neurological disorders, and neuropathic pain.

19. The compound of claim 8, wherein said compound is capable of inhibition of the kappa (κ) opioid receptor.

20. The compound of claim 8, wherein the opioid receptor agonist is selected from the group consisting of morphine, methadone, codeine, diacetyl morphine, morphine-N-oxide, oxymorphone, oxycodone, hydromorphone, hydrocodone, meperidine, heterocodeine, fentanyl, sufentanil, levo-acetylmethadol, alfentanil, levorphanol, tilidine, diphenoxylate, hydroxymorphone, noroxymorphone, metopon, propoxyphene, and the pharmaceutically acceptable salts thereof.

21. A compound having a formula selected from the group consisting of: 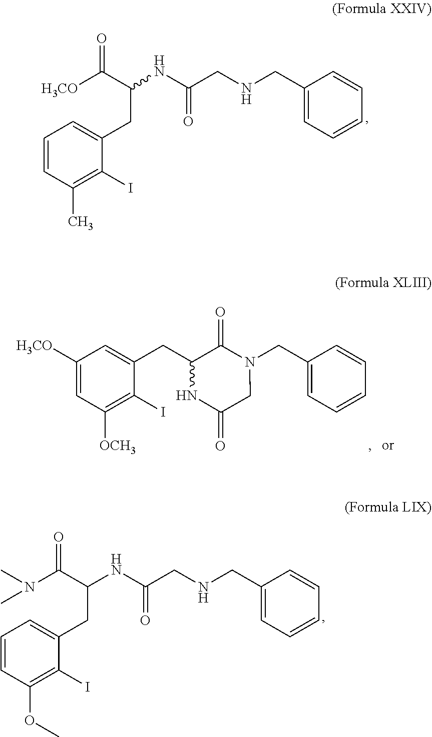 [SMILES: COC(=O)C(Cc1cccc(C)c1I)NC(=O)CNCc1ccccc1 ; COc1cc(CC2NC(=O)CN(Cc3ccccc3)C2=O)c(I)c(OC)c1 ; COc1cccc(CC(NC(=O)CNCc2ccccc2)C(=O)N(C)C)c1I]
or combination thereof.

22. The compound of claim 21, comprising a pharmaceutical composition having a therapeutically effective amount of said compound or a pharmaceutically acceptable salt thereof or isotopic variants thereof, stereoisomers or tautomers thereof.

23. The compound of claim 21, wherein said compound is capable of having at least 50% of the administered amount cross the blood-brain barrier (BBB) of a patient.

24. The compound of claim 21, wherein said compound is capable of acting peripherally to selectively inhibit KOR.

25. The compound of claim 21, wherein said compound is effective to treat addiction, alcohol dependence, opioid abuse treatment, neurological disorders, and neuropathic pain.
